(19) United States
(12) Patent Application Publication
(10) Pub. No.: US 2023/0121271 A1
(43) Pub. Date: Apr. 20, 2023

(54) METHODS FOR PRECISE AND BIAS-FREE QUANTIFICATION OF CELL-FREE DNA

(71) Applicant: Chronix Biomedical, Irvine, CA (US)

(72) Inventors: Ekkehard Schütz, Göttingen (DE); Julia Beck, Göttingen (DE)

(21) Appl. No.: 17/914,933

(22) PCT Filed: Mar. 25, 2021
(86) PCT No.: PCT/US2021/024231

§ 371 Date: Sep. 27, 2022

Related U.S. Application Data
(60) Provisional application No. 63/001,062, filed on Mar. 27, 2020.

(51) Int. Cl. C12Q 1/686; C12Q 1/6848

(52) U.S. Cl. CPC C12Q 1/686; C12Q 1/6848

(57) ABSTRACT

This invention provides a method of quantifying the amount of diagnostic cell-free DNA (cfDNA) in a cfDNA sample from a subject. Specifically, the method comprising quantifying the amount of a diagnostic cfDNA in a cfDNA sample from a subject using two or more PCR reactions, wherein the diagnostic cfDNA deviates from germline DNA of the subject for differentiating cfDNA between the shortening or lengthening of the individual's germline-originating cfDNA and the shortening or lengthening of the target diagnostic cfDNA from cells of interest that are present in the subject, such as cells from a tumor, a fetus (placenta) or a transplanted organ.

### CROSS-REFERENCE TO RELATED APPLICATIONS

This application claims priority benefit of U.S. Provisional Application No. 63/001,062, filed Mar. 27, 2020, which is incorporated by referenced for all purposes.

### BACKGROUND

Detection and quantification of target-specific cell-free DNA (cfDNA) has become an important diagnostic tool for subjects having various medical conditions, including, e.g., cancer, pregnancy, or transplantation. Any medical situation that leads to the presence of cells in a subject in which the cellular genome is different from the germline genome of the subject is suitable for cfDNA analysis for evaluating the presence and quantity of such deviating DNA in the body fluids of an individual for diagnostic and/or prognostic purposes.

As one example, PCR technologies evaluating donor vs recipient alleles based on polymorphisms in the population may be employed to analyze cfDNA in transplant patients.

Typically, the data in these analyses are presented as percentage of total circulating cfDNA in the plasma, or in any given body fluid. Any enrichment for such targets (e.g., PCR-based, whether multiplexed or not) is sensitive to the fragmentation pattern of the DNA. However, a high fragmentation due to degradation is one main characteristic of cfDNA. Thus, a cfDNA fragment that is shorter than the designed PCR amplicon will not be detected. Further, if a PCR-amplicon has, e.g., half of the size of a cfDNA fragment, the detection rate will be only 50% of the amount of the cfDNA that is actually present in the fluid being analyzed because the fragmentation is quasi-random. Thus, the total amount of cfDNA is inferred based a summation of randomly degraded DNA with an individual length and length distribution, which can be very different in various samples.

U.S. Patent Application Publication No. 20170327869 describes a method to quantify the total amount of cell-free DNA in body fluids, such as plasma, with amplification-based technology by taking into account the degree of overall fragmentation (degradation) of cfDNA in an individual sample. This publication shows the importance of the individual differences that occur in in terms of shortening of the cell-free DNA for analytics, in particular for quantification purposes of the total cfDNA. Described therein is a method to correct for the resulting measurement bias, based on the assessment of the individual overall fragmentation of all cfDNA by performing PCR reactions that generate amplicons of different lengths and comparing the yields to provide a correction factor. The general relationship between a certain average DNA length in a sample and the yield of a PCR with a certain amplicon length can be described by the following formula:

$$DNA observed = DNA present \times \frac{DNA length - Amplicon length}{DNA length} = DNA present \times ( 1 - \frac{Amplicon length}{DNA length} )$$

The method described in U.S. Patent Application Publication No. 20170327869 thus provides the ability to more accurately quantify the number of cfDNA molecules in the presence of any individual mean-length of the inferred dDNA. In the case of an admixture of DNA stemming from the germline and any given target DNA (e.g. a transplanted organ, a tumor or a fetus) there is no distinction between those two moieties, nor is it needed for the intended purpose.

At present there is evidence that cfDNA from, e.g., cancer (Jiang et al., Proc Natl AcadSci USA 112:E1317-25, 2015; Mouliere et al., PLoS One 6:e23418, 2011), placenta (Fan et al, Proc NatlAcad Sci USA 105:16266-71,20018; Chan et al., Clin Chem 50:88-92, 2004); or organ grafts (Lui et al., Clin Chem 48:421-7, 2002) tends to be more shortened than the cfDNA stemming from germline. For the sake of detection (not exact quantification) the developers of cfDNA test have designed assays with amplicons as short as technically possible. The underlying rationale for this assay design is that using short amplicons is sufficient to generate a non-biased assessment of the percentage of target cfDNA (of total cfDNA). Recommendations arising after having observed the effect of shortening of target cfDNA in PCR reaction are to use amplicons of, e.g., <100 base pairs (bp) (Mouliere et al., supra, 2011) or <143 bp (Barrett et al., Clin Chem 58:1026-32, 2012), or other values. For example, it has been proposed to use an averaging of multiple reference genes using a GeNorm approach to more reliably estimate the total cfDNA quantity (Devonshire, et al., AnalBioanal Chem 406:6499-512, 2014). It has also been described that using such PCR-based estimation of target cfDNA leads to similar results (Bruno et al., Clin Chem 60:1105-14, 2014), if the amplicon sizes are comparable, using either real-time PCR (Bruno et al, 2014) or next generation sequencing (Natera) (Zimmermann et al, PrenatDiagn 32:1233-41, 2012) for the analysis, which was not the case if a longer PCR was used as a comparison. Notably, the accuracy of values generated using a “short amplicon” approach is compromised, as the calculated values would only be accurate if (i) the length of both DNa moieties is the same or (ii) the amplicon length is zero). Thus, there is methodological bias in such methods.

As a further example, the detection of rejection by evaluating donor-derived cell-free DNA (dd-cfDNA) quantification has become a valuable diagnostic tool. Transplant rejection is characterized by several events, of which activation of immune-system with marginalization of leukocytes from the bone marrow into the blood stream is one. This is important, because the majority of cfDNA found in plasma originates from circulating white blood cells (WBCs) (Sun et al., Proc NatlAcad Sci USA 112:E5503-12, 2015). Accordingly, the percentage of donor-derived cfDNA can fluctuate depending on the amount of DNA originating from the host transplant recipient WBCs. It is also known that the cfDNA from WBCs has a fragmentation with a characteristic dominant peak of about 167 bp and minor peaks at multiples of 167, with a typical mean fragment length of about 250 bp. This value was derived from over 4,000 measurements of cell-freeDNA fragmentation (Beck et al., US20170327869). But, the cfDNA is inter-individually variable. For instance in patients from 2 weeks up to 5 years after kidney transplantation a median value of 260 bp was found for the average cfDNA length with a 95% confidence interval of 213 bp to 508 bp. However, cfDNA from “deeper compartments” than circulating WBC (e.g., organ tissue) can be substantially shorter, as reported, e.g., for placental or tumor cfDNA or from organ grafts.

### BRIEF SUMMARY OF ASPECTS OF THE DISCLOSURE

Certain aspects of the disclosure are summarized below. The invention is not limited to the particular embodiments described in this summary of the disclosure.

The present application, in based at least in part, on the recognition of the technical bias of percentage measurements of target cfDNA that originates from cells present in a subject that do not contain the normal germline DNA of the subject. Thus, in one aspect, provided herein are methods and kits for assessing differences in fragmentation in germline vs. target cfDNA in a subject. The method provides striking improvements in the ability to accurately determine target DNA concentrations (e.g., measured as copies/ml) and/or percentages of target DNA.

Rejection of organs especially in the case of kidney occurs in different regions of the graft. If primarily mediated by T-lymphocytes, T-cell mediated rejection (TCMR), the affected area is mainly the tubular interstitium, whereas if humoral antibodies cause the rejection the damaged side is mainly the vascular endothelium. In contrast, DNA released during a necrosis of a transplant, which can occur early after engraftment and often leads to a delayed graft function, has longer fragments, which might even be longer that the usually observed recipient cfDNA. The consequences would be an overestimation of such necrotic cfDNA percentage. Thus, in a further aspect, provided herein are illustrative data demonstrating that fragmentation is more extensive in TCMR and moderate in ABMR, whereas the length is above of the length of recipient cfDNA in a necrotic episode and improved quantification methods that take into consideration such differences in fragmentation.

The same quantification bias explained above will occur in any such measurement, if it is targeted towards tumor specific cfDNA (ctDNA) or fetal cfDNA or any other cfDNA that is present in low amounts and presents a situation in which the length deviates from the genome-originating cfDNA, which represents the majority of the denominator of percentage calculation.

In a further aspect, provided herein is a method of differentiating cfDNA between the shortening or lengthening of the individual's germline-originating cfDNA and the shortening or lengthening of the target diagnostic cfDNA from cells of interest that are present in the subject, such as cells from a tumor, a fetus (placenta) or a transplanted organ. Thus, in one aspect, described herein is a method to improve quantitation of diagnostic cfDNAs in a sample, e.g., from serum, plasma, or blood. The method comprises employing PCR reactions that generate amplicons of different lengths and comparing the results of the percentage yield between those PCRs in the germline-originating cfDNA compared to the diagnostic cfDNA. In one embodiment, the PCR reactions are employed in a multiplex reaction. In one embodiment, by computing the intercept of a linear correlation with amplicon length as an independent variable and the percentage yield of diagnostic cfDNA as a dependent variable, a more accurate value of diagnostic cfDNA can be calculated (interpolation to an amplicon length of zero bp).

### BRIEF DESCRIPTION OF THE DRAWINGS

FIG. 1: Illustrative data from a digital PCR showing calculation of donor fragment size in cfDNA from a kidney transplant patient with biopsy provide TCMR.

FIG. 2: Illustrative data from a digital PCR showing calculation of donor fragment size in cfDNA from a kidney transplant patient with chronic active ABMR.

FIG. 3: Illustrative data from a digital PCR showing calculation of donor fragment length in cfDNA from a kidney transplant patient with a chronic/acute mixed type of rejection.

FIG. 4: Illustrative data from a digital PCR showing calculation of donor fragment length in cfDNA from a kidney transplant patient with acute necrotic damage.

FIG. 5 shows the raw measured dd-cfDNA percentages from the sample analyzed for FIG. 4.

FIG. 6 shows an in silico simulation of percentage quantification bias in dependency of amplicon and targeted cfDNA length. upper line, Target 3: 310 bp; middle line. Target 2: 139 bp; lower line, Target 1: 113 bp

FIG. 7 shows an in silico simulation of concentration (copies/ml) quantification bias in dependency of amplicon and targeted cfDNA length. upper line, Target 3: 310 bp; second line from top, Target 4: 235 bp (host); third line from top, Target 2: 139 bp; lower line, Target 1: 113 bp

FIG. 8 depicts an in silico simulation of the effect of different length of diagnostic PCRs on the diagnostic capabilities to detect rejections. upper line, Target 3: 150 bp (reference); middle line. Target 2: 139 bp (ABMR); lower line, Target 1: 113 bp (TCMR)

FIG. 9 depicts the results from a patient after bone marrow transplant in which the patient that has a relatively low amount of cfDNA.

FIG. 10 depicts the result from a patient after bone marrow transplant in which the patient has a with relatively high total amount of cfDNA.

FIG. 11 provides data illustrating the effect of control samples with the same length and different length of target (minor fraction) and host (major fraction) cfDNA on the results for percentage determinations using a digital PCR.

FIG. 12 shows the mean allele frequencies and ratio of short/long amplicons for samples in Example 11.

### DETAILED DESCRIPTION

The term “cell-free DNA” or “cfDNA” as used herein means free DNA molecules of 25 nucleotides or longer that are not contained within any intact cells. In the context of the current invention, “cfDNA” is typically evaluated in human blood, e.g., can be obtained from human serum or plasma.

Generally, cfDNA is fragmented. As used herein, the “proportion of amplifiable diagnostic DNA” or “fraction of amplifiable germline DNA” in a cfDNA sample refers to the amount of diagnostic or germline DNA in a sample that can provide an amplified product of a size of interest.

In the context of the present invention “germline-originating” cfDNA refers to cfDNA in a sample from a subject that is germline DNA from that subject. “Target” or “diagnostic” cfDNA refers to DNA originating from cells in the subject that do not contain germline DNA and thus have DNA that deviates in sequence from the germline DNA of the subject. Such cells can be from another subject, e.g., transplant tissue from a donor, or fetal cells, or can be cells in the subject that deviate from germline, e.g., cancer cells or other cell containing mutations, chromosomal abnormalities, and the like.

A “graft” as used herein refers to tissue material, from a donor that is transplanted into a recipient. For example, a graft may be from liver, heart, kidney, or any other organ.

The term “primer” refers to an oligonucleotide that acts as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The primer includes a “hybridizing region” exactly or substantially complementary to the target sequence, preferably about 15 to about 35 nucleotides in length. A primer oligonucleotide can either consist entirely of the hybridizing region or can contain additional features which allow for the detection, immobilization, or manipulation of the amplified product, but which do not alter the ability of the primer to serve as a starting reagent for DNA synthesis. For example, a nucleic acid sequence tail can be included at the 5′ end of the primer that hybridizes to a capture oligonucleotide.

The term “probe” refers to an oligonucleotide that selectively hybridizes to a target nucleic acid under suitable conditions. A probe for detection of the biomarker sequences described herein can be any length, e.g., from 15-500 bp in length. Typically, in probe-based assays, hybridization probes that are less than 50 bp are preferred.

The term “target sequence” or “target region” refers to a region of a nucleic acid that is to be analyzed and comprises the sequence of interest, e.g., a region containing a SNP biomarker, or a mutation of interest.

As used herein, the terms “nucleic acid,” “polynucleotide” and “oligonucleotide” refer to primers, probes, and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. Oligonucleotides for use in the invention may be used as primers and/or probes.

A nucleic acid, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide of the invention can contain one or more modified, non-standard, or derivatized base moieties, including, but not limited to, N6-methyl-adenine, N6-tert-butyl-benzyl-adenine, imidazole, substituted imidazoles, 5-fluorouracil, 5 bromouracil, 5-chlorouracil, 5-iodouracil, hypoxanthine, xanthine, 4-acetylcytosine, 5 (carboxyhydroxymethyl)uracil, 5 carboxymethylaminomethyl-2-thiouridine, 5 carboxymethylaminomethyluracil, dihydrouracil, beta-D-galactosylqueosine, inosine, N6 isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2 thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785, each of which is incorporated herein by reference in its entirety. Furthermore, a nucleic acid, polynucleotide or oligonucleotide can comprise one or more modified sugar moieties including, but not limited to, arabinose, 2-fluoroarabinose, xylulose, and a hexose.

A “unique sequence” as used herein is a sequence that is free of repeated DNA that can be localized to a single site on a genome. For example, SNP loci for amplification for transplant analysis are localized to a unique sequence on the genome.

As used herein, the singular forms “a,” “an” and “the” include plural referents unless the context clearly dictates otherwise. Thus, for example, reference to “a molecule” includes a plurality of such molecules, and the like.

### INTRODUCTION

The present disclosure, provides, at least in one aspect, methods of more accurately quantifying cfDNA of an interrogated target, e.g., from a tumor, fetus, or a transplanted organ, by differentiating cfDNA that originates from cells of interest in a subject, e.g., cancer cells, fetal, cells, or transplanted tissue. The methods employ at least two PCR reactions that generate amplicons of different lengths to assess the degree of fragmentation of germline DNA present in cfDNA compared to that of non-germline DNA present in cfDNA. Because germline cfDNA, largely originating from white blood cells of the subject does not exhibit as much fragmentation as cfDNA in the patient that deviates from germline, the methods as described herein provide improved quantification of non-germline cfDNA, whether determined as a percentage or as a concentration.

Analysis of cfDNA in Patients

In one illustrative embodiment, cfDNA is analyzed in transplant patients to evaluate graft rejection. As noted above, the majority of cfDNA present in plasma is known to originate from circulating white blood cells (WBCs). As described herein, the degree of fragmentation differs for cfDNA from the subjects WBCs compared to fragmentation of cfDNA from rejected transplant tissue. For example, as shown in the EXAMPLEs section, fragmentation is more pronounced, i.e., there are shorter fragments in TCMR compared to ABMR and the length is greater than the length of recipient cfDNA in a necrotic episode.

In the present invention, at least two PCR reactions are performed with primers that generated amplicons of different lengths. The yields are then compared in the differing PCR reactions to provide an improved quantification of the percent or concentration of donor-derived cfDNA.

Primers are selected that amplify a target region that comprises a sequence that is specific to an allele that is present in graft tissue from a donor but not present in the recipient. In some embodiments, the target region comprises a SNP allele that is present in a donor, but not the recipient subject. In some embodiments, the primers employed in the PCR reactions to generate amplicons of different length are selected to amplify the same target region, but to generate amplicons of different lengths. In some embodiments, one of the primers in each primer set shares at least partial sequence identity such that the sequences in the target region to which the primers hybridize overlap. Thus, for example, a forward primer of a primer set to generate a shorter amplicon may hybridize to a nucleic acid sequence that at least partially overlaps with the nucleic acid sequence to which the forward primer that generates the longer amplicon hybridizes. In some embodiments, the primer sets for each amplicon share a common primer that hybridize to the same target sequence. For example, a forward primer of a primer set to generate a shorter amplicon may be the same primer sequence as the forward primer to generate a longer amplicon. In typical embodiments, the primers in each primer set are both different and hybridize to different sequences in the target region.

In some embodiments, the amplicons that different in length are generated from two non-overlapping target regions. For example, one primer set may be selected to amplify a first target region that comprises a sequence, e.g., a first SNP that differs in the donor and the recipient whereas a second primer set to generate a second amplicon that differs in size from the first amplicon may be selected to amplify a second target region that does not overlap with the first and comprises a second sequence, e.g., a second SNP, that is different in the donor compared to the correspondence sequence in the recipient.

The bias based on different target cfDNA lengths is not limited to dd-cfDNA. The same quantification bias will occur in any such measurement, if it is targeted towards tumor specific cfDNA (ctDNA) or fetal cfDNA or any other DNA that is present in low amounts where the length deviates from the length of WBC-derived cfDNA, which as noted above, represents the majority of the denominator of percentage calculation.

In a further illustrative embodiments, tumor-specific cfDNA (ctDNA) is quantified. For example, quantification of cancer cell derived ctDNA in plasma is already implemented for several so-called somatic cancer mutations in the medical field. Examples are mutations in the EGFR gene in non-small cell lung cancer (NSCL) or mutations in the KRAS gene in adenocarcinomas (e.g. colon or pancreas). The vast majority of detection and quantification methods takes advantage of a PCR-based targeting of the region(s) of such genes that are commonly mutated in cancer. In particular, if such an assay is used to monitor the amount of ctDNA in plasma, a precise quantification of the ctDNA is important in longitudinal surveillance to ensure that the medical interpretation of observed dynamic changes under therapy is based on reliable quantification. At present all available quantification methods do not take the described methodological bias into account, assuming the length of germline cfDNA and the length of ctDNA is constant, even under chemotherapy or immunotherapy or immune therapy.

Thus, in some embodiments cfDNA analysis is performed on patient that has a cancer. The cancer can be any kind of cancer so long as cancer cells have a genome that comprises mutations that distinguish the genomes of the cancer cells from the germline genome of the patient. Thus, for example, a patient may have lung cancer, e.g., non-small cell lung cancer, breast cancer, colorectal cancer, ovarian cancer, prostate cancer, pancreatic cancer, bladder cancer, liver cancer, head and neck cancer, a neurological cancer, e.g. a glioblastoma; or a hematopoietic cancer, e.g., a leukemia or lymphoma of any type.

PCR primers to generate amplicons of different length can be selected as described above for analysis of transplant patient cfDNA. Thus, primers are selected that amplify a target region that comprises a sequence that is specific to tumor DNA, e.g., a mutation such as a KRAS mutation and not present in the germline DNA of the patient.

In some embodiments, the primers employed in the PCR reactions to generate amplicons of different length are selected to amplify the same target region, e.g., a region that comprises a mutation, but to generate amplicons of different lengths. In some embodiments, one of the primers in each primer set shares at least partial sequence identity such that the sequences in the target region to which the primers hybridize overlap. Thus, for example, a forward primer of a primer set to generate a shorter amplicon may hybridize to a nucleic acid sequence that at least partially overlaps with the nucleic acid sequence to which the forward primer that generates the longer amplicon hybridizes. In some embodiments, the primer sets for each amplicon share a common primer that hybridize to the same target sequence. For example, a forward primer of a primer set to generate a shorter amplicon may be the same primer sequence as the forward primer to generate a longer amplicon. In typical embodiments, the primers in each primer set are both different and hybridize to different sequences in the target region.

In some embodiments, the amplicons that different in length are generated from two non-overlapping target regions. For example, one primer set may be selected to amplify a first target region that comprises a sequence, e.g., a first mutation that is present in cancer cells, but not the patient germline DNA, whereas a second primer set to generate a second amplicon that differs in size from the first amplicon may be selected to amplify a second target region that does not overlap with the first and comprises a second sequence, e.g., a second mutation associated with the cancer cells, but is not present in the patient germline DNA.

In a further illustrative embodiments, prenatal testing can be performed by evaluating cfDNA in a body fluid, e.g., serum or plasma, from a pregnant subject, a pregnant human subject. Non-invasive prenatal testing (NIPT) interrogates the fetal DNA, released by the placenta into the mother's blood stream. A major application of NIPT is screening for numeric chromosomal abnormalities of a fetus, such as trisomy 21 (the Down syndrome). One technology employed is based on using frequently mutated loci to differentiate fetal cfDNA from the mother's fraction of cfDNA. This technique is essentially the same as the evaluation of cfDNA in transplant patients and is therefore prone to the same analytical bias as described herein. PCR reactions are designed to amplify regions that comprises a sequence that differs in fetal vs maternal DNA (see, e.g., Zimmerman et al., Prenat Diagn 32:1233-1241, 2012). In Zimmerman et al., in order to calculate the ploidy of diagnostic cfDNA, the method employs modelling of the observed minor allelic frequencies (i.e., the fetal fraction) against expected allelic frequencies of different ploidy states. This method is based on a high-dimensional multiplex PCR followed by sequencing and is reported to use amplicons as short as possible, e.g., around 60 bp.

As noted above, primers are selected that provided amplicons of different lengths. In some embodiments, the amplicons differ by 10 base pairs in length. In typical embodiments, the amplicons different by at least 15 base pairs in length. In some embodiments, the amplicons differ by at least 20 base pairs in length, or may different aby at least 25, at least 30, at least 35, at least 40, at least 45, or at least 50 base pairs in length. In typical embodiments, the primers are selected that generate amplicons that differ by no more than 100 or 150 base pairs in length, or no more than 200 base pairs in length. In some embodiments, the difference in amplicon sizes is in the range from 10 to 200 base pairs in length. In typical embodiments, the amplicons differ in size from about 20 to about 150 base pairs in length.

In some embodiments, a control PCR reaction may be performed that further comprise “spike-in” DNA, i.e., control DNA added to the starting sample obtained from a patient, e.g., a blood, plasma, or serum sample, to control for the efficiency of extraction of the cfDNA from the patient sample.

### Amplification of DNA

reactions are performed on cfDNA obtained a sample, typically blood, serum, or plasma, from a subject. A “subject” or a “patient” in the context of this invention is any individual that is to be evaluated using a diagnostic cfDNA assay in which cfDNA that deviates from germline is quantified. In typical embodiments, the patient is a human. In other embodiments, the patient is a mammal, e.g., a murine, bovine, equine, canine, feline, porcine, ovine, caprine, or a primate.

In some embodiments, digital PCR is performed in which a limiting dilution of the sample is made across a large number of separate PCR reactions so that most of the reactions have no template molecules and give a negative amplification result. Those reactions that are positive at the reaction endpoint are counted as individual template molecules present in the original sample in a 1 to 1 relationship. (See, e.g., Kalina et al. NAR 25:1999-2004 (1997) and Vogelstein and Kinzler, PNAS 96:9236-9241 (1999); U.S. Pat. Nos. 6,440,706, 6,753,147, and 7,824,889; each incorporated by reference.) In some embodiments, a digital PCR may be a microfluidics-based digital PCR. In some embodiments, a droplet digital PCR may be employed.

### Quantification

The amplicons obtained for each of the PCR reactions can be evaluated using any known technology, including, for example, digital droplet PCR, high throughput sequencing technology, or a hybridization assay that employs capture probes.

In some embodiments, DNA sequence and analysis are used to analyze amplicons obtained from the PCT reactions. For example, DNA sequencing may be accomplished using high-throughput DNA sequencing techniques. Examples of next generation and high-throughput sequencing include, for example, massively parallel signature sequencing, polony sequencing, 454 pyrosequencing, Illumina (Solexa) sequencing with HiSeq, MiSeq, and other platforms, SOLiD sequencing, ion semiconductor sequencing (Ion Torrent), DNA nanoball sequencing, heliscope single molecule sequencing, single molecule real time (SMRT) sequencing, MassARRAY®, and Digital Analysis of Selected Regions (DANSR™)

Any technology that employs targeted hybridization (e.g., primer oligonucleotides or hybrid capture oligonucleotides) for selection of any genomic position can be used to evaluate the amounts of each amplicon generated. One of skill understands that in such an embodiment, if two capture probes located upstream and downstream directly adjacent to the target mutation are not used, the same problem of underestimating the target would occur for those fragments, which lack the hybridization region of a single capture probe.

The methods provided herein correct for the bias in previous methods of target cfDNA quantification that do not fully account for varying fragmentation in germline cfDNA vs. the non-germline cfDNA being quantified.

In some embodiments, the percentage yield is compared for the PCR reactions that generate amplicons of different lengths. In some embodiments, a concentration of cfDNA of interest is determined using PCR reactions as described herein to improve quantification.

In one embodiment, by computing the intercept of a linear correlation with amplicon length as an independent variable and the percentage yield of diagnostic cfDNA as a dependent variable, an absolute concentration of dd-cfDNA can be calculated (interpolation to an amplicon length of zero bp). For example, a linear regression is performed with the length of the individual amplicon as independent and the measured dd-cfDNA percentage as dependent variable. As explained above, the average length of the subject germline cfDNA is determined. The determined average length is used to calculate the amplifiable fraction of germline cfDNA (θcƒDNA) to correct the denominator of the percentage value for the diagnostic non-germline cfDNA. The y-value for the regression is measured non-germline-cfDNA×θcƒDNA and the interpolation of the regression line into zero provides an accurate non germline cfDNA percentage.

In a first step the average length of the total cfDNA needs to be determined. In a second step the amplifiable fraction of the total cfDNA (θcƒDNA) is calculated for each amplicon length used in the sample. The resulting θcƒDNA value is multiplied with the measured percentage value of the target (e.g dd-cfDNA in case of transplantation) and are plotted vs. the used amplicon length, which usually would be performed with computer assistance. The interpolation of the values into zero bp (e.g the intercept of a regression line) gives the true value of the target. This can be deduced from the equation (U.S. Patent Application Publication No. 20170327869) shown in the Background section of the present application, since only with 0 bp the term solves to 1. The same equation can be rearranged for DNA length as follows:

$$DNA measured = DNA present \times ( 1 - \frac{Amplicon length}{DNA length} ) > DNA length = - \frac{Amplicon length}{\frac{DNA measured}{DNA present} - 1}$$

Therefore, in one embodiment, the following formula can be applied to each result of two or more used PCRs with different amplicon lengths on an individual sample:

$$mean ddcfDNA length = - \frac{Amplicon length}{( measured ddcfDNA \% \times θ cfDNA ) / ( intercept ddcfDNA \% ) - 1}$$

### Computer Analysis

In some embodiments, the present invention provides systems related to the above methods of the invention. In one embodiment the invention provides a system for analyzing circulating cell-free DNA, comprising: (1) a sample analyzer for executing the method of analyzing germline cf DNA and diagnostic non-germline cfDNA in a patient's blood, serum or plasma using at least two PCR reactions that generate amplicons of different lengths to calculate the amplifiable fraction of the germline cfDNA and diagnostic non-germline cfDNa in the sample as described above; (2) a computer system for automatically receiving and analyzing data obtained in step (1) to calculate the fraction of amplifiable germline DNA in the cfDNA and the fraction of amplifiable diagnostic non-germline DNA in the sample.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 8, Windows™ 7,Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™ or Microsoft™ Explorer™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out the analysis and correlating functions as described above. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

### EXAMPLES

The following examples are provided by way of illustration only and not by way of limitation. Those of skill in the art will readily recognize a variety of non-critical parameters that could be changed or modified to yield essentially similar results.

The following examples describes the development of an improved quantitative cfDNA assay that provides accurate quantification of cf

### Example 1: Assessment of cfDNA from a Kidney Transplant Patient with TCMR

Digital PCR using different amplicon lengths was used to evaluate cfDNA from a kidney transplant patient with biopsy-proven TCMR. Assays were performed as described (Beck et al, Clin Chem 59:1732-1741, 2013; U.S. Patent Application Publication No. 20160115541). In brief, digital PCR reactions interrogating SNPs that are highly abundant in the human population were used, where both occurring alleles are separately quantified using different fluorophores for allele-specific hydrolysis probes. To generate results with host (transplant recipient) and donor-specific amplicon length, informative PCRs with different amplicon lengths were selected. The effective amplicon length is defined herein as the shortest 3′ portion of each primer with an at least 75% binding at the PCR conditions added to the captured DNA fragment. Even though primer hybridization can be considered to follow a two-state hybridization it still follows the law of mass action. Thus, the proportion of primer/template in double strand (dsDNA) formation vs the proportion in single state formation is a continuum over the annealing temperature at otherwise constant conditions (Marky, and Breslauer, Biopolymers 26 (1987) 1601-20, Schutz and v.Ahsen, Anal Biochem 2009; 385: 143-52), where the steepness of the hybridization/temperature curve is a function of van′t Hoff enthalpy. The percentage of primer/template in dsDNA state, which is the state to initiate the amplification of the 5′-restricted primer sequence, can therefore be calculated based on its thermodynamic properties. For example, if primer 1 has 22 bp and it would still bind with the fifteen 3 'bases with 75% binding (dsDNA) these 15 bp would be used. If primer 2 has 25 bp and it would still bind with the fourteen 3′ bases with 75% binding these 14 bp would be used. That said, from the total amplicon length 9 bp and 7 bp would be subtracted to calculate the effective amplicon length in the PCR. In theory, only two different amplicon sizes with a lbp difference would needed to generate a true target value, if there would be no measurement error. But the technical imprecision of PCR itself (e.g., binding dynamics as outlined above), the technical error of the counting or quantification method used, and the statistical error when detecting trace amounts (which follows the Poisson distribution) has to be considered to gain precise and reliable results. It is known in the field that the accuracy of a regression line is best at the point of average of x-values/average of y-values (center point of data). From both directions from the center point the confidence interval gets broader. In addition, assuming two clusters of data (e.g., two different length of PCR) with a given dispersion, the prediction of the intercept has a lower confidence interval, the higher the span of the x-value is. The invention as described herein is using the interpolation of the measured data into the point of an amplicon length of zero, which is the intercept of a regression line, which needs to have a minimized error. This has two consequences: the lowest x-value should be as near to zero as possible and the highest x-value should be as high as possible. The lowest PCR amplicon length is dictated by technical limits; but the longer amplicon needs to be carefully optimized in length. As shown herein, the longer an amplicon is, the lower is the efficiency of the amplification, because the longer the amplicon is, the higher is the chance of template strand-breaks occurring between the two primers. As a consequence, for fragmented DNA, the number of amplifiable (and amplified) target molecules decreases with increasing amplicon length of any primer-based detection method. This leads to an increased counting error since the error can be estimated as the square root of the count. For example, if 100 DNA molecules were present and all are counted, the error is 10%. If only 30% were amplified, the error would be 18%. Such an increased dispersion of data in the y-direction would lead to an increased error of the intercept of the regression line. That said, the error would be smaller if the length of the cfDNA is increasing. As a further consideration, the biological error needs to be accounted for. A cfDNA—if, e.g., found to have an average length of 250 bp, has a variability (Gaussian distribution), which can be individually variable. In using extensive error simulations taking all the variables from above into account, we found that an optimal range of PCR length would be between 45 bp for the short PCRs and 75-85 bp for the longest PCRs. The lowest number of PCRs should be four for both length and can be as high as several thousands. The confidence limit of the intercept does not change substantially if more than 200 PCRs are used, e.g. the difference between 200 and 2,000 is minimal, since the biological error is the limiting factor.

A linear regression was performed with the length of the individual amplicon ad independent and the measured dd-cfDNA percentage as dependent variable. For this calculation the average length of the host cf-DNA is required and to be known and can be determined, e.g., as described in U.S. Patent Application Publication No. 20170327869). The determined average length is used to calculate the amplifiable fraction of host cfDNA (θcƒDNA) to correct the denominator of the percentage value for dd-cfDNA. The y-value for the regression is measured dd-cfDNA×θcƒDNA and the interpolation of the regression line into zero showed a true dd-cfDNA percentage value of 1.9%. Without accounting for the degradation effect the values was calculated as 0.73%. The mean length of the fragmented kidney DNA was assessed as 113 bp by applying the following formula to each result of the four used PCRs:

$$mean ddcfDNA length = - \frac{Amplicon length}{( measured ddcfDNA \% \times θ cfDNA ) / ( intercept ddcfDNA \% ) - 1}$$

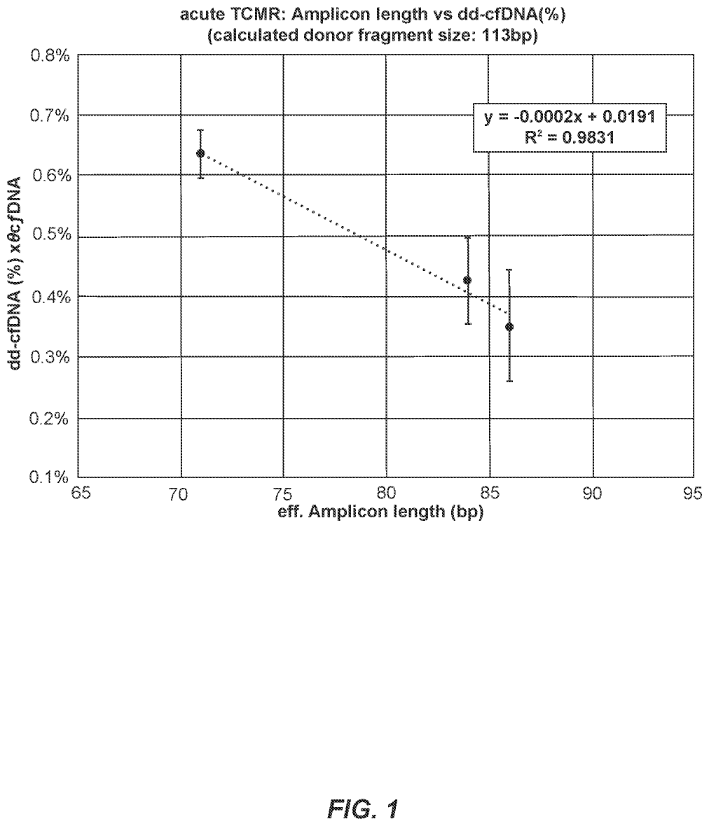

The average of the results for each data point was calculated and is shown in FIG. 1.

### Example 2. Assessment of cfDNA from a Kidney Transplant Patient with Chronic Active ABMR

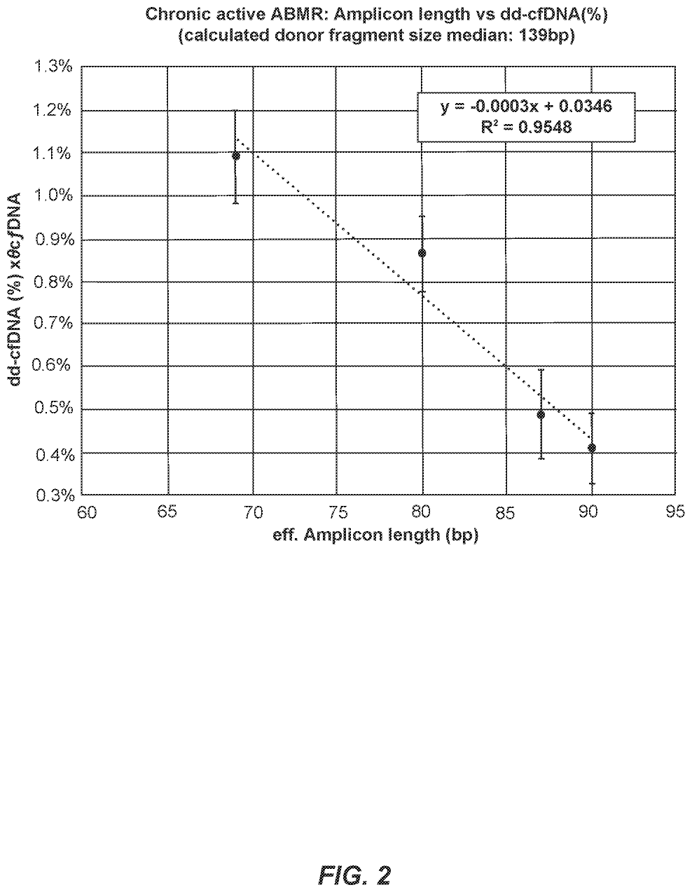

Digital PCR using different amplicon lengths was used to evaluate cfDNA from a kidney transplant patient with chronic active ABMR. A linear regression was performed using the same method as given in example 1 and the interpolation of the regression line into zero showed a real dd-cfDNA percentage value of 3.5% as the true percentage value. Without accounting for the degradation effect the values was calculated as 1.1%. The mean length of the fragmented kidney dd-cfDNA was assessed as 139 bp (FIG. 2) by the formula given in Example 1.

### Example 3. Assessment of cfDNA from a Kidney Transplant Patient with Chronic/Acute Mixed Type Rejection

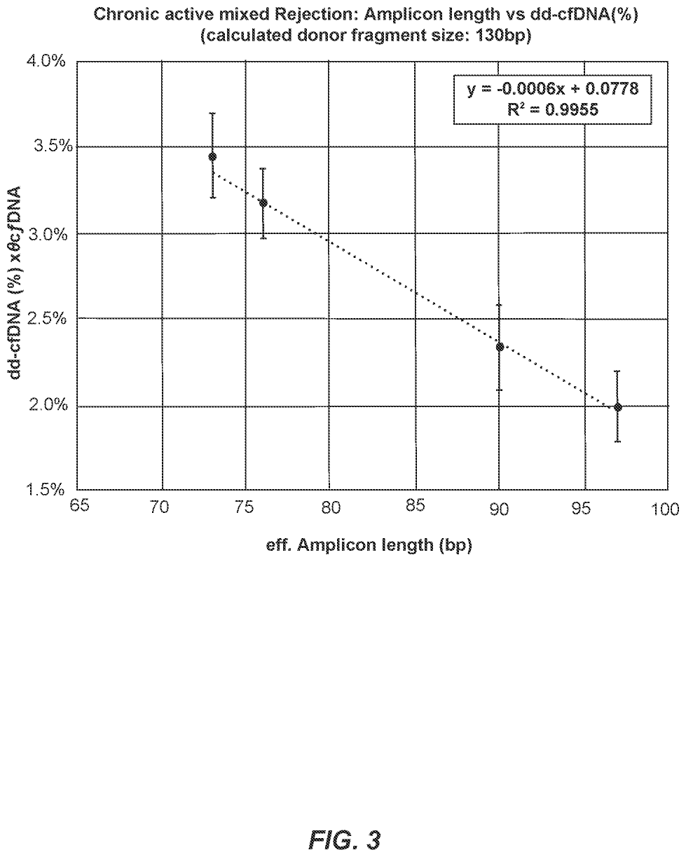

Digital PCR using different amplicon lengths was used to evaluate cfDNA from a kidney transplant patient with an chronic/acute mixed type rejection. A linear regression was performed using the same method as given in Example 1 and the interpolation of the regression line into zero showed a real dd-cfDNA percentage value of 7.8%. Without accounting for the degradation effect the values was calculated as 4.3%. The mean length of the fragmented kidney dd-cfDNA (FIG. 3) was assessed as 130 bp by the formula given in Example 1.

### Example 4. Assessment of cfDNA from a Kidney Transplant Patient with Acute Necrotic Damage

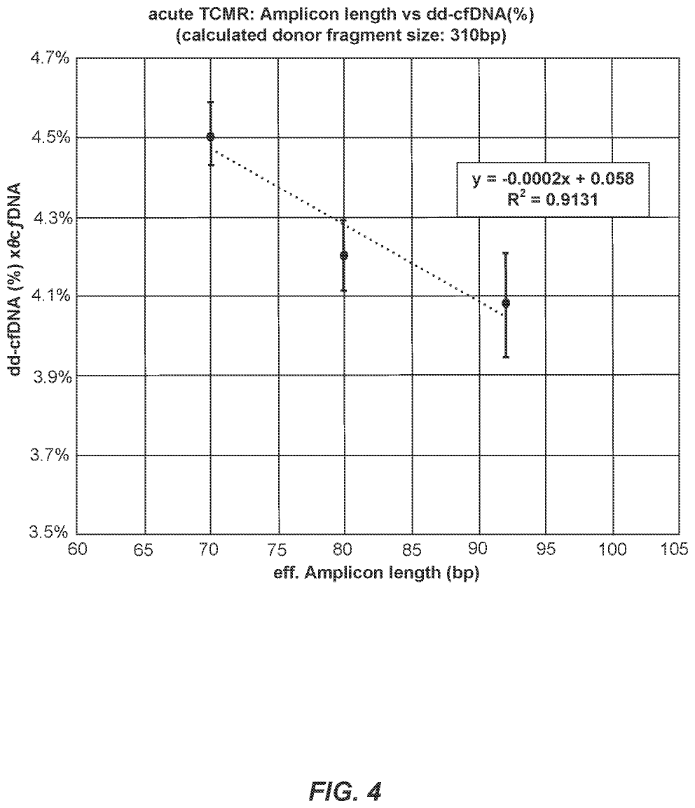

Digital PCR using different amplicon lengths was used to evaluate cfDNA from a kidney transplant patient with acute necrotic damage. A linear regression was performed using the same method as given in Example 1 and the interpolation of the regression line into zero showed a real dd-cfDNA percentage value of 5.8%. Without accounting for the degradation effect the values was calculated as 7.2%. The mean length of the fragmented kidney dd-cfDNA (FIG. 4) was assessed as 310 bp by the formula given in Example 1.

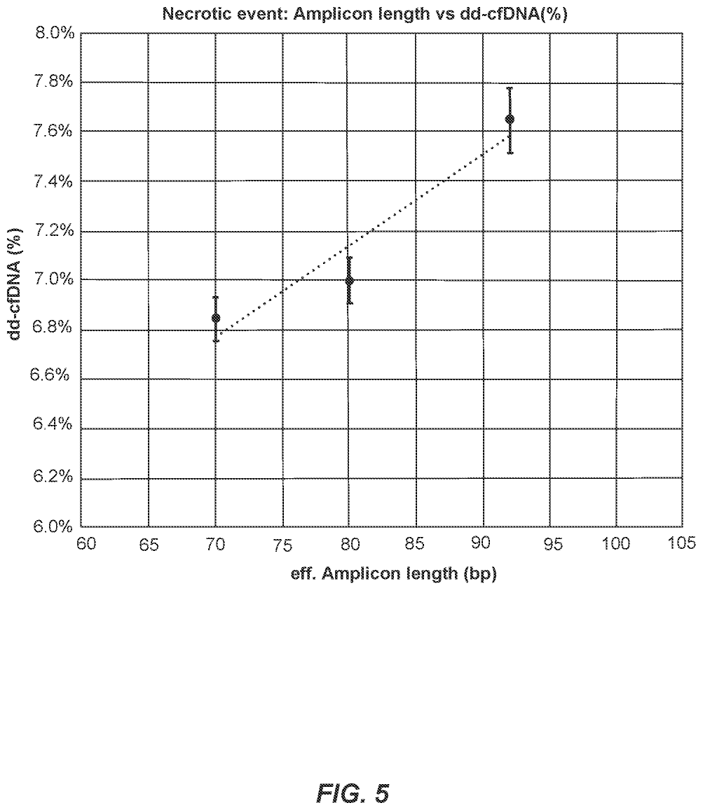

FIG. 5 shows the raw measured dd-cfDNA percentages of this sample. It is seen that the effect of amplicons length on percentage has a positive correlation, due to the fact that the dd-cfDNA is longer in such a necrotic dd-cfDNA release than the mostly apoptotic leukocyte stemming cfDNA of the host. An observation of this inverse correlation in a sample indicates a high likelihood of necrotic graft damage.

In using samples with biopsy proven isolated TCMR, ABMR and acute tubular necrosis (ATN) we have used the method described to verify the general concept of differentially shortened dd-cfDNA under the different clinical conditions. For each sample the mean length of the amplifiable cfDNA from the host (WBC) was determined as described1. The derived θcƒDNA for the quantification ddPCR was the converted to the mean amplifiable cfDNA length, by rearranging equation 1 for DNA length. The resulting value was used in equation 2 for the calculation of the true dd-cfDNA values by interpolating the values generated with different length dPCRs to an amplicon size of zero as shown in the examples above. The results are given in Table 1.

TABLE 1
Average values of samples drawn under different biopsy proven
conditions are given for each group. All samples showed isolated
biopsy results no mixed pathologies were used. 1) Lengths of
short PCRs were 47-51 bp; lengths of long PCRs were 85-91 bp.

| | Short | Long | | | |
|---|---|---|---|---|---|
| Source | PCR¹ % | PCR² % | | True % | Length |
| KTx | dd-cfDNA | dd-cfDNA | n | dd-cfDNA | in bp |
| TCMR | 0.93 | 0.67 | 3 | 1.22 | ~124 |
| ABMR | 0.91 | 0.89 | 4 | 0.97 | ~178 |
| Stable pat. | 0.28 | 0.24 | 4 | 0.30 | ~187 |
| ATN | 1.45 | 1.72 | 4 | 1.28 | >500 |
| Host (WBC) | n.a. | n.a. | 1,270 | n.a. | ~254 |

### Example 5. In Silico Simulated Effect of Amplicon and Target Region Length on Estimated Percentage of Target DNA

In a broader aspect the effect of amplicon and targeted region length can be simulated in silico. For this, 1,000,000 simulation were computed per data point assuming the WBC-derived host cfDNA of having a length with an average of 254 bp using R. All given target lengths were assumed to have a biological variation leading a standard deviation of 15% of the average values being Gaussian distributed.

The effect on diagnostic PCRs with different amplicon lengths for a true value of 1.5 is shown in the following table, which gives the measured values (±standard deviation) in dependence of mean fragment length of the target DNA.

TABLE 2

| true cfDNA: 1.5 | | cfDNA 120 bp | cfDNA 175 bp | cfDNA 700 bp |
|---|---|---|---|---|
| 60 | bp Amplicon | 1.00 ± 0.08 | 1.3 ± 0.06 | 1.80 ± 0.05 |
| 80 | bp Amplicon | 0.75 ± 0.13 | 1.19 ± 0.09 | 1.96 ± 0.08 |
| 100 | bp Amplicon | 0.46 ± 0.14 | 1.07 ± 0.13 | 2.15 ± 0.12 |
| 110 | bp Amplication | 0.32 ± 0.13 | 0.99 ± 0.15 | 2.26 ± 0.15 |
| 120 | bp Amplicon | 0.19 ± 0.12 | 0.90 ± 0.17 | 2.40 ± 0.19 |
| 130 | bp Amplicon | 0.10 ± 0.09 | 0.81 ± 0.19 | 2.55 ± 0.23 |

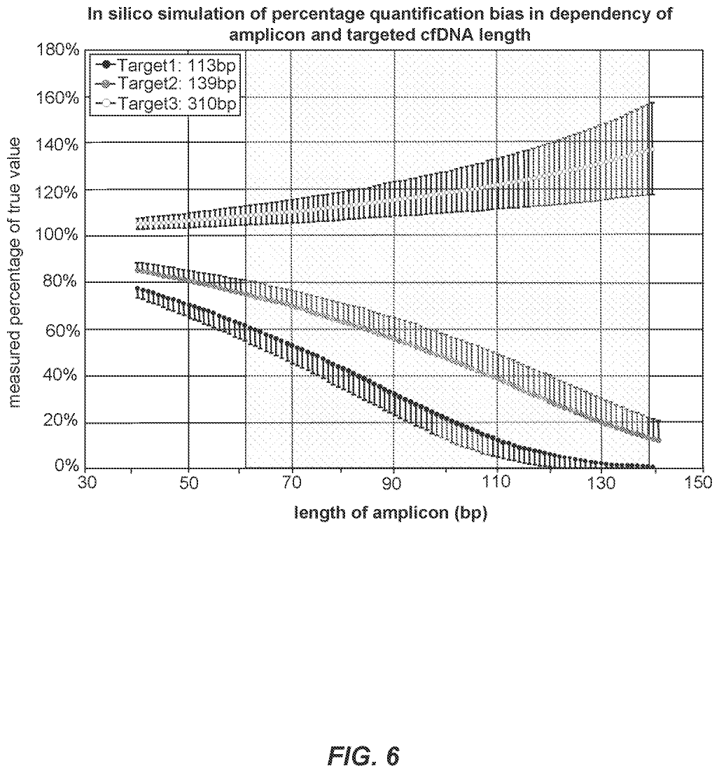

FIG. 6 shows the simulated effect on the estimated percentage of a target cfDNA, as percentage of the true value in a sample. Error bars represent the standard deviation, which is calculated using the Gaussian multiplicative error propagation from the standard deviations of the numerators (Targets) and denominator (254 bp). The shaded area represents the PCR amplicon length that are used in PCR-based dd-cfDNA assays at present. It is apparent that an underestimation of dd-cfDNA is unavoidable in rejection where the dd-DNA is substantially shortened as shown in Table 1. The underestimation is increasing with longer PCR amplicons. The same holds true for the overestimation of dd-cfDNA in a clinical event where necrosis dominates, as the cfDNA is less shortened than the host cfDNA from white blood cells.

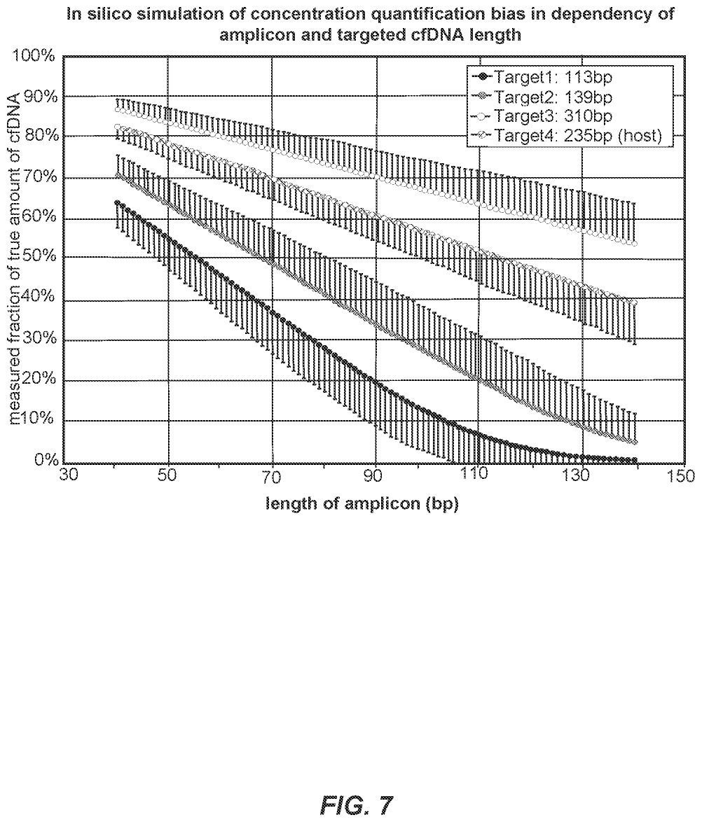

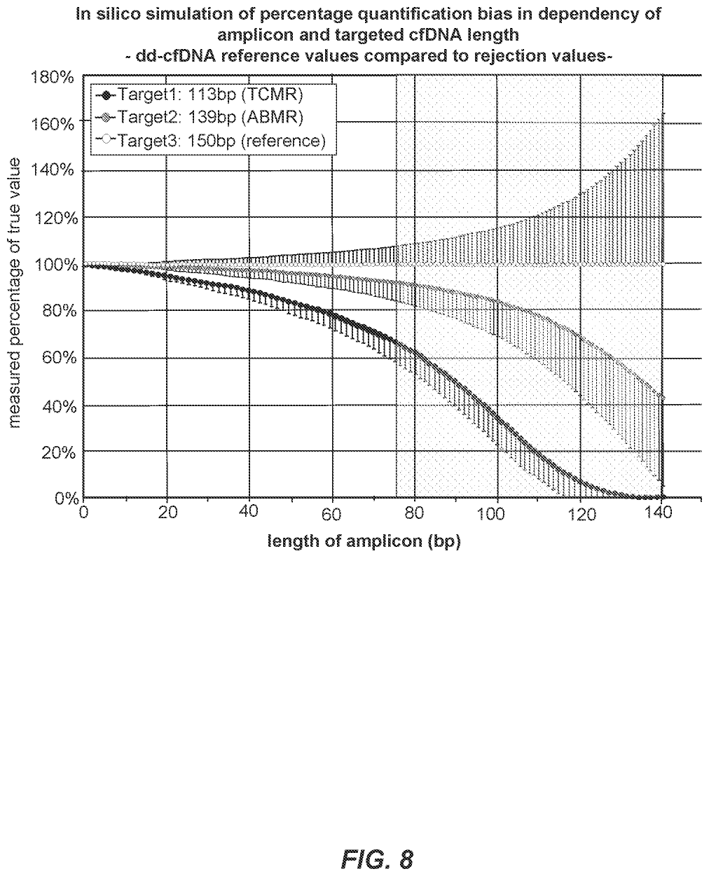

The bias described above (dependency of cfDNA length and amplification length) applies not only to percentage estimations, but will also lead to an underestimation of copies/ml of target cfDNA in any given circumstance per se. Using the same assumptions as given above for the percentages the results of the effect simulations are shown in FIG. 7. It is evident that the under-estimation of cfDNA by PCR has a clear dependency on both fragment length and amplicon length. Further, it is also apparent that a direct quantification of only the target DNA (e.g. in copies/mL) is biased for any sort of fragmented DNA. This leads to certain consequences concerning diagnostic performance to detect rejections. Assuming the dd-cfDNA of a healthy kidney graft has a mean length of about 185 bp, such dd-cfDNA would naturally serve to define the amount of dd-cfDNA circulating in the recipient without being an indicator of rejection or any other graft damage. Usually the biological variability is determined in a larger group of patients without any clinical signs of graft damage (e.g. clinically stable) or from a group of patients where a biopsy shows no pathologic signs). Against such a background of normal biologic variance—often referred to as reference range (or in a one-sided clinical setting the upper limit of a reference range)—the results of a patient sample is compared and e.g. called positive if the measured value exceeds the reference limit. FIG. 8 depicts the effect of different length of diagnostic PCRs on the diagnostic capabilities to detect rejections. The data are calculated as given for FIG. 6.

Several effects can be determined from the data in FIG. 8:

a) The broadness of an estimated reference range increases with increasing amplicon lengths, which can be explained by the hypergeometric effect occurring if only a few percent of the real abundant dd-cfDNA is used for the quantitative estimation. Since dd-cfDNA is only present in trace amounts (e.g. median 25cp/mL) in clinically stable kidney recipients8, if using cfDNA extracted from 4 mL patient plasma a PCR with an amplicon length of e.g. 110 bp would only be able to detect 22 of the 85 copies and the 95% confidence limits would be 13 copy to 31 copies; the coefficient of variation (CV) is thus 21%. In contrast a 60 bp PCR would yield a CV of 14%; a theoretical amplification with a 10 bp amplicon would have a CV of 11%. Such a higher technical variance will add to the real biological variance, leading to a broader apparent reference range.
b) The longer the used amplicons are, the more an underestimation of rejection stemming dd-cfDNA would occur (Lui et al, Clin Chem 2002, 48:421-7, 2002; Duque-Afonso et al., Clin Biochem 52:137-41, 2018). This would be more extreme in TCMR cases with more shortened dd-cfDNA as shown in Table 1. Again, using the example of a 110 bp diagnostic amplicon it is evident that the dd-cfDNA in a TCMR case would only be determined to be higher than in the reference group, if it is at least 3-fold elevated in the plasma of the patient. This might explain a recently reported effect of virtually lower dd-cfDNA in TCMR patients compared to biopsy proven stable kidney recipients, with an assay using long (>100 bp) PCRs for such diagnostic purposes (Huang et al., Am J Transplant 19:1663-70, 2019).

In particular, the deteriorating effects on the diagnostic use of cfDNA in transplantation explained under section b) above can be avoided by using two or more PCR reactions that provide amplicons of different lengths with a subsequent extrapolation to a 0 bp amplicon.

### Example 6. Assessment of cfDNA in Liver and Heart Transplant Patients

In liver transplantation the variability of dd-cfDNA is the widest of all transplanted solid organs (Schutz et al., PLoSMed 2017, 14:e1002286, 2017). The percentage of dd-cfDNA can be as low as 5% and above 50% in severe liver graft damage. The same principles of detection bias by PCR detailed in the earlier examples apply to liver and heart transplant recipients, when using single length allele-specific detection as described e.g. in Beck et al., Clin Chem. 12:1732-41, 2013.

Table 3 shows bias correction for examples of patients after heart and liver transplantation in various different clinical situations. The procedure used was the same as described in Example 1.

TABLE 3

| | | Uncorrected | Corrected | Average length |
|---|---|---|---|---|
| | Clinical status | dd-cfDNA | dd-cfDNA | dd-cfDNA |
| HTX | ISHLT 2R | 1.7% | 4.4% | 127 bp |
| | 24 hr after engraftment | 8.9% | 13.6% | 238 bp |
| LTX | Sever necrosis | 58.2% | 94.8% | 255 bp |
| | Severe rejection | 36.8% | 68.0% | 198 bp |

### Example 7. Assessment of cfDNA in Bone Marrow Transplant Patients

Bone marrow transplantation (BMT) is a more complex situation, since apart of rejection of the donor cells, also a graft verus host disease (GvHD) can occur. The overall knowledge about cfDNA in BMT is scarce, whereas a broad fragmentation pattern of cfDNA seems to occur^{4,7}. We have observed a highly variable amount of total cfDNA in patients after BMT over three orders of magnitude up to >200,000 cp/mL, which was significantly higher (P<0.01) and more variable (P<0.0001) than before BMT. We also noted a high abundance of shortened cfDNA with average cfDNA length<200 bp. These observations have a significant influence on the estimation of both donor- and recipient-derived cfDNA after BMT, when when using single length allele-specific detection as described, e.g., in Beck et al., 2013, supra.

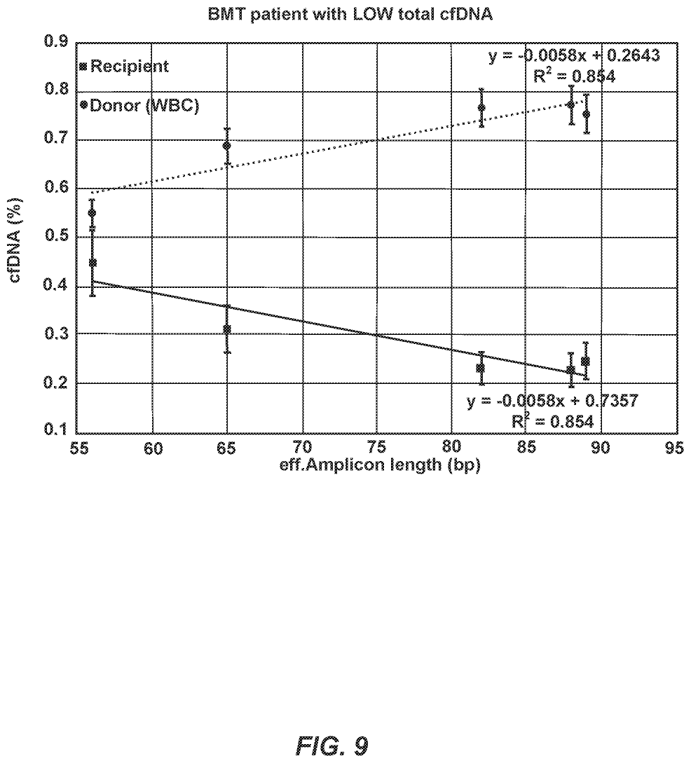

FIG. 9 depicts the result from a patient (case 1) after BMT with relatively low amount of cfDNA (24,000 cp/mL). The average in the BMT patients was 67,000 cp/mL. The estimated percentage of recipients cfDNA was 29% without any corrections. The allel-specific evaluation of the cfDNA length revealed that the recipient cfDNA tends to be longer than the leukocyte cfDNA is this case. The total circulating amount of recipient cfDNA was calculated with 18,000 cp/mL. As a general observation if cfDNA from a deep compartment is present in high amounts, it tends to be of longer fragment sizes.

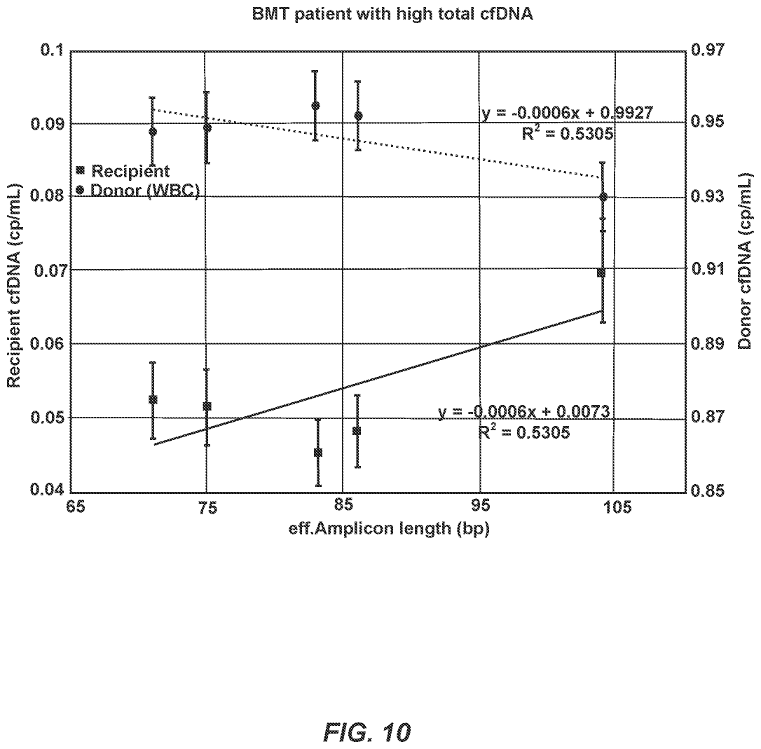

FIG. 10 depicts the result from a patient (case 2) after BMT with relatively high amount of cfDNA (162,000 cp/mL). The estimated percentage of recipients cfDNA was 4% without any corrections. The allele-specific evaluation of the cfDNA length revealed that the recipient cfDNA tends to be shorter than the WBC cfDNA, which seems longer than the usually observed leukocyte cfDNA is this case. The total circulating amount of recipient cfDNA was calculated with 1,180 cp/mL.

The effect of the correction for length differences of recipient and donor cfDNA is given in Table 4:

TABLE 4

| Recipient (cp/mL) | Corrected | Uncorrected |
|---|---|---|
| Case1: | 18,000 | 7,200 |
| Case2: | 1,180 | 8,200 |

### Example 8. Assessment of cfDNA from Patient with Pancreatic Cancer

Although it is well known that cell-free DNA from solid malignant tumors can be shorter than the WBC cfDNA (Jiang et al., Proc Natl Acad Sci USA, 112:E1317-25, 2015; Mouliere et al., PLoS One 6:e23418, 2011), the extent of such shortening has not been comprehensively investigated, particularly for patients undergoing therapy. PCR-based methods are used, however, to monitor the therapeutic response in patients by serial quantification of cell-free tumor DNA (ctDNA).

Samples from patients with pancreatic ductal adenocarcinomas being treated with Folfirinox as a chemotherapeutic agent were used to evaluated the potential shortening of ctDNA. A commercially available digital droplet PCR assay for the somatic tumor mutation KRAS.pG12D was used, together with two additional ddPCRs for the same locus (Primer1.F: GTATCGTCAAGGCACTCTT, Primer1.R: CCTGCTGAAAATGACTGAAT and Primer2.F: CGTCCACAAAATGATTCTGA, Primer2.R: ATAAGGCCTGCTGAAAATGA). The detecting hydrolysis probes for the latter two assays were: (Wildtype: HEX-TCTTGCCTACGCCACCAG-BHQ1, MutationGl2D: FAM-TAGTTGGAGCTGATGGCG-BHQ1). The amplification primers generated amplicons of different sizes. The amplicon lengths were between 57 and 107 bp. The same calculations as described in Example 1 were used to determine the percentage of KRAS-mutated ctDNA for two patients (Patient 1, two samples; Patient 3, five samples). The results are provided in Table 5.

TABLE 5

| | Patient 1 | Patient 1 | Patient 3 | Patient 3 | Patient 3 | Patient 3 | Patient 3 |
|---|---|---|---|---|---|---|---|
| KRAS Assay | Sample 1 | Sample 2 | Sample 1 | Sample 2 | Sample 3 | Sample 4 | Sample 5 |
| 57 bp* | 5.50% | 1.84% | 4.73% | 1.54% | 1.54% | 1.51% | 1.00% |
| 74 bp | 4.20% | 2.10% | 4.24% | 1.36% | 1.15% | 1.21% | 0.80% |
| 107 bp | 4.2% | 1.0% | 3.3% | 0.9% | <0.15% | 0.5% | <0.15% |
| Corrected | 5.8% | 2.4% | 5.4% | 1.86% | 2.12% | 2.01% | 1.3% |
| Pearson's r | −0.9389 | −0.9133 | −0.9999 | −0.9996 | −1.00 | −0.9998 | −1.00 |
| ctDNA length | 169.0 | 142.7 | 154.0 | 140.9 | 110.7 | 119.0 | 120.6 |
| SD | 26.4 | 30.1 | 0.4 | 1.7 | NA | 1.1 | NA |

*Commercial ddPCR according to manufacturer documentation

It can be seen that the differences observed in cfDNA length lead to variability in quantification, with lower amounts of mutated K-RAS observed for longer amplicon sizes. In addition, the ctDNA lengths can vary substantially, with an average ranging from 111 bp to 169 bp; even within one patient the observed span was 111 bp to 141 bp. The corrected values determined in accordance with the present invention were respectively higher than the values measured with the commercial assay. Such differences in ctDNA length within on patient under therapy can lead to wrong interpretations of the clinical course. For an example, an apparently falling percentage of K-RAS-mutated ctDNA determined using an assay that does not adequately control for degree of fragmentation in the sample may be due to a higher degree of fragmentation of the ctDNA in a later sample compared to an earlier sample in the same patient. Thus, a decrease in concentration of mutated K-RAS observed over time in such an assay may be due to increased fragmentation, not an actual decrease in the concentration of K-RAS-mutated cfDNA.

One particular issue is the use of artificial controls for PCR-based assay quality control purposes. All available controls are made from artificial samples, where the target and host (patient noncancerous genomic DNA) is sheared to simulate the fractionation of cfDNA. No such control, in particular the commercially available controls, takes the differentially shortening of target and host cfDNA into account. Thus, a useful artificial sample to control the resulting bias should be manufactured in such a way that the target is of shorter length than the artificial host DNA.

### Example 9: Assessment of Fetal Fractions in Maternal Plasma

Five maternal plasma samples were analyzed for fetal cfDNA using the methods described above. Table 6 shows the differences in the measured fetal fraction using amplicons with a mean length of 53 bp and amplicons with a mean length of 93 bp. The true value of the fetal fraction were calculated by linear regression and using the formula provided in the “Quantification” section of the DETAILED DESCRIPTION of the present application.

The average length of the fetal cfDNA fragments varies between 117 and 207 bp.

TABLE 6
Results for five maternal plasma samples

| Sample | NIPT1 | NIPT2 | NIPT3 | NIPT4 | NIPT5 |
|---|---|---|---|---|---|
| Average 53 bp Amplicon | 2.73% | 12.33% | 6.07% | 2.83% | 3.26% |
| Average 93 bp Amplicon | 2.06% | 10.1% | 3.37% | 1.77% | 2.41% |
| True Value | 3.18% | 13.15% | 8.20% | 3.57% | 3.75% |
| Length fetal fraction | 158 bp | 207 bp | 117 bp | 117 bp | 122 bp |
| Ratio length | 62% | 72% | 48% | 48% | 52% |
| (fetal/maternal) | | | | | |

### Example 10: Artificial Quality Control Samples

Genomic DNA samples of two individuals were sheared by ultrasound to two different fragment lengths: Individual A to ˜135 bp (average fragment length) and Individual B to ˜240 bp (average fragment length). The size of the fragments after shearing was determined as described in U.S. Patent Application Publication No. 20170327869.

Three dilutions with the genomic DNA sheared to 135 bp as minor fraction into the genomic DNA sheared to 240 bp as major fraction were used (Minor Short 1-3). One additional sample was diluted the opposite way (Minor Long) and one control sample consisted of genomic DNAs sheared to the same average length of ˜235 bp (Equal Length).

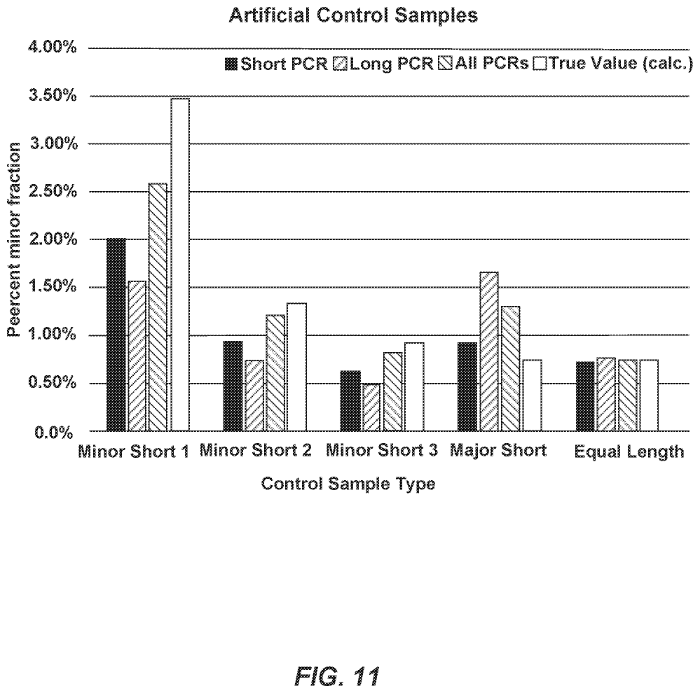

The samples were preamplified in two different multiplex PCR reactions with average fragment lengths of 50 bp and 89 bp (primer sequences and positions of targeted SNPs set forth in Table 7). Four ddPCRs with short amplicons (average length: 41.5.bp) and four PCRs with long amplicons (average length: 81 bp) were subsequently carried out to determine the minor allelic fraction for each sample. FIG. 11 shows the effect of control samples with the same length and different length of target (minor fraction) and host (major fraction) cfDNA on the results on percentage determinations using a digital PCR.

TABLE 7
Primers for short/long preamplification

| | | | | SNP | Ampli- |
|---|---|---|---|---|---|
| | | Primer | | position | con |
| Primer (forward) | Sequence | (reverse) | Sequence | (HG19) | Length |
| Short Amplicon Primers | | | | | |
| (Multiplex Preamplification) | | | | | |
| S038_short_P5.for | GAGGACTTGGCACAGGTGCTC | S038_short_ | GCTCTCTAAGTGGAGACGG | chr2:21755 | 47 |
| | | P7.rev | GTCC | 1954 | |
| S046_short_P5.for | TCTGCCCTGGGAGAGAAAGAACAA | S046_short_ | GGTCTCCTTTCATTTCCCCAA | chr16:1326 | 52 |
| | | P7.rev | ATGC | 5483 | |
| S043_short_P5.for | CACAACTTCCCTAAGGGACTGACAT | S043_short_ | TCTCATCTGTAAAAGGGGGT | chr10:7156 | 52 |
| | | P7.rev | GGTG | 7011 | |
| S048_short_P5.for | GGACTCTTGCCAGTTTCCATGACAA | S048_short_ | AACACGCCGGGAGCCCTGC | chr16:8770 | 47 |
| | | P7.rev | | 5868 | |
| S050_short_P5.for | GTTTCTGAGGCTCGGTTTTCGCT | S050_short_ | GGTCGCCGAATAGTCCATTT | chr1:47716 | 49 |
| | | P7.rev | CAC | 65 | |
| S053_short_P5.for | AAGGCAGGACTTCTCCACCCA | S053_short_ | CCCAAGGTGGCTTTTGAGAC | chr8:10418 | 47 |
| | | P7.rev | AAG | 7996 | |
| S055_short_P5.for | ACATCCATGGAATACTTCTCAGCAA | S055_short_ | CAGCATAGTTCTCTGGAAAT | chr18:3769 | 57 |
| | CA | P7.rev | TCATCCA | 560 | |
| S057_short_P5.for | TTGCACACTCACGTTTGGGATACT | S057_short_ | AGAACCCCAGTAAGGAATG | chr6:31045 | 52 |
| | | P7.rev | GAGAAA | 574 | |
| S058_short_P5.for | CCAAAGTGGTAGGATTACAGGCAT | S058_short_ | CAAACTTGGCAAGGCACGG | chr2:47854 | 49 |
| | GA | P7.rev | T | 988 | |
| S059_short_P5.for | GTCTGAATTTTACTCCTGGCTCTGC | S059_short_ | GGGAAGGGACTTGATTACA | chr8:20808 | 57 |
| | A | P7.rev | TAGCTTATC | 908 | |
| S066_short_P5.for | TCAGTTCCTTCACCACTGTTATTTGT | S066_short_ | GGAGCTAGGATATTGCTAG | chr11:8082 | 57 |
| | TC | P7.rev | AGTGGAG | 5491 | |
| S067_short_P5.for | GCGAAGTTGGAGAGGGTTGGGG | S067_short_ | AGTACCAAGACCCCGACCCT | chr19:3009 | 48 |
| | | P7.rev | TAA | 7268 | |
| S068_short_P5.for | CACGCCCTTCTGAGTCCCACA | S068_short_ | TGTTGGAAAGGACAGCAGG | chr8:10539 | 47 |
| | | P7.rev | AGAC | 3816 | |
| S070_short_P5.for | ATGCTGGCATACCCTCCTGTACT | S070_short_ | GCGTGTGGACAGTGAAGGT | chr2:22436 | 48 |
| | | P7.rev | GTG | 3562 | |
| S077_short_P5.for | GCTATTGTATGCTCTATGCTCAGCA | S077_short_ | TGATTCACCTGCACTGCTTCC | chr16:1036 | 52 |
| | CA | P7.rev | C | 640 | |
| S079_short_P5.for | AGGCGCCGGCAGCAGGTGC | S079_short_ | GCTCTCTAAGCATTAGGCAT | chr9:13851 | 48 |
| | | P7.rev | TACTGC | 8114 | |
| S082_short_P5.for | GGAAGGAAGTGCAGGAGGGCTG | S082_short_ | CCAGTCAAGGCCTCTGCTCT | chr7:42380 | 47 |
| | | P7.rev | CA | 77 | |
| S084_short_P5.for | CATGATGCTCACAAAGACATTCCTC | S084_short_ | GGAGTGGAGGCCAGAAGTC | chr18:4481 | 50 |
| | C | P7.rev | CC | 9849 | |
| S085_short_P5.for | GAAACATCTAGACGCGCAATGACA | S085_short_ | GGCGGAATCGGGAGGCCAC | chr5:14089 | 47 |
| | C | P7.rev | | 2643 | |
| S086_short_P5.for | CAGCCGGGAGGAAAGAAACCTTT | S086_short_ | AGGTGCTTGTGAGGATTAAC | chr1:25889 | 52 |
| | | P7.rev | TGACAT | 422 | |
| S087_short_P5.for | AGGCTATAAACACTGGGATGGGGG | S087_short_ | AGCTGAGTTTCAAGGCTTGT | chr10:1309 | 51 |
| | | P7.rev | ACAC | 52994 | |
| S090_short_P5.for | GGAAAAGGAATTCCTGGCAGAGGG | S090_short_ | CTCCCTGCCTATGCTCAGGC | chr22:2546 | 48 |
| | | P7.rev | A | 1040 | |
| S092_short_P5.for | AGGTCCTCAAGCTGTGCAACTG | S092_short_ | AGACCTTCAAACCACCTTGT | chr5:14961 | 48 |
| | | P7.rev | GGC | 1793 | |
| S094_short_P5.for | GCTTAGGTTTGAGGTAGCACAGAG | S094_short_ | GAACCCAGCCACAGCTGCA | chr10:1857 | 48 |
| | GA | P7.rev | | 1215 | |
| S097_short_P5.for | CCCTCCTCCATCAGGTGCTGG | S097_short_ | GCTTCTGATCCTGCAGGGAA | chr18:8563 | 46 |
| | | P7.rev | GA | 171 | |
| S102_short_P5.for | GTTGTGGCCTTATCTTTGGCCCTA | S102_short_ | CCTCTTGTTTGAGGCACATC | chr13:2759 | 52 |
| | | P7.rev | CTACA | 7230 | |
| S105_short_P5.for | CTTTATAGGGGAGGAGTGGAGGA | S105_short_ | ATTCCTGTGCCACTGGGCTG | chr6:31106 | 48 |
| | GG | P7.rev | | 893 | |
| S108_short_P5.for | ACTCCCCACATATATGCTCCCCA | S108_short_ | GTCTGCATGGTCCCAGCTGG | chr12:1132 | 46 |
| | | P7.rev | | 02967 | |
| Long Amplicon Primers | | | | | |
| (Multiplex Preamplification) | | | | | |
| S038_long.F | TCAATCCTCACAACTTCCCTAAGGG | S038_short. | AGTGGGAGGGAGGTACAGT | chr2:21755 | 90 |
| | | R | GA | 1954 | |
| S046_long.F | tccagcagaggaaatagtacttgc | S046_short. | agccacctggtctcctttca | chr16:1326 | 90 |
| | | R | | 5483 | |
| S043_long.F | GTCTCTGGGGGTCTGTTGGCC | S043_short. | AGAGGAAGGACTCCCAGGG | chr10:7156 | 100 |
| | | R | GG | 7011 | |
| S048_long.F | GATCAACTCCTGAAGAGACTCCGT | S048_short. | AGGGAGGGATGGAGAGGG | chr16:8770 | 91 |
| | | R | AC | 5868 | |
| S050_long.F | TCTTGTCGAGGCTGCCCTGAAAGG | S050_short. | ACAGAGCCGGCCGGTCGC | chr1:47716 | 91 |
| | | R | | 65 | |
| S053_long.F | CAAAGAGCTCRAACCCCAAG | S053_short. | TGTGGGCAAGGCAGGACT | chr8:10418 | 68 |
| | | R | | 7996 | |
| S055_long.F | TGGTTAAACTGTAGTACATCCATGG | S055_short. | ACCTTTTGGGACTGGCTTTC | chr18:3769 | 98 |
| | A | R | T | 560 | |
| S057_long.F | CAGCCTCTGGTTCCAGGCCT | S057_short. | ggagaatcccagaagcaggctga | chr6:31045 | 107 |
| | | R | | 574 | |
| S058_long.F | gccaccttagcctcccaaag | S058_short. | AGGGTGACTGTATTAATTAT | chr2:47854 | 87 |
| | | R | TGTTCAAACT | 988 | |
| S059_long.F | AGAAAGAAAGAAGCAGGGAAGGG | S059_short. | TGGAGCTAAAATGAGCCTGC | chr8:20808 | 92 |
| | AC | R | GT | 908 | |
| S066_long.F | ACCCTGACCCTCAGTTCCTT | S066_short. | AAGAGCCCTTATAAGGTGTG | chr11:8082 | 98 |
| | | R | AGAAA | 5491 | |
| S067_long.F | ATGAAGAGTAAGCGGGGCCG | S067_short. | CGGACCCATTTCACCCACCA | chr19:3009 | 86 |
| | | R | | 7268 | |
| S068_long.F | GCCTCTGCCATATCCTCAC | S068_short. | AGGTCGGATGTTGGAAAGG | chr8:10539 | 71 |
| | | R | | 3816 | |
| S070_long.F | TGGCCCAGTTAGAAGGTGTGGA | S070_short. | CGGCCACCCATCCTGGAGAT | chr2:22436 | 97 |
| | | R | | 3562 | |
| S077_long.F | GGGCCTCAGTTCTAGACGAGT | S077_short. | GTTTCCGTGAAGTAGGCGCT | chr16:1036 | 96 |
| | | R | | 640 | |
| S079_long.F | CAGGGAGTGCTTTACTGAGGC | S079_short. | ACTCAAACACGGAGCTGGG | chr9:13851 | 96 |
| | | R | C | 8114 | |
| S082_long.F | TTTGCACTTGACGCACCAGC | S082_short. | CCGAGGCAGAGGAAGGAAG | chr7:42380 | 79 |
| | | R | TG | 77 | |
| S084_long.F | CCCCAAACTAAGTACCTAATCACTC | S084_short. | CCAAGGGGAGCATCCACCAT | chr18:4481 | 96 |
| | GT | R | | 9849 | |
| S085_long.F | ACACACACACACGCAATTCGG | S085_short. | ATGAGCTGAGGTGGGTGCT | chr5:14089 | 94 |
| | | R | G | 2643 | |
| S086_long.F | GTCTCCCTCCCCAAAGGTGC | S086_short. | GCCAACCTCAAGGGGCAGT | chr1:25889 | 95 |
| | | R | T | 422 | |
| S087_long.F | GGCATCTGAATTCAAGCTTTGGTC | S087_short. | TTCTTCTAGTTGGTCTGGTA | chr10:1309 | 94 |
| | | R | GGCT | 52994 | |
| 5090_long.F | GTTGAACGTCCACAGAAGGA | S090_short. | GGCTGCTCAGCCTCCCTG | chr22:2546 | 76 |
| | | R | | 1040 | |
| S092_long.F | TTTATTTAAATGACTGTCCAGGTC | S092_short. | TTTCACAGACCTTCAAACCA | chr5:14961 | 73 |
| | | R | C | 1793 | |
| S094_long.F | CTGGGGCAGAGTGGAGAGTC | S094_short. | ATCCACCTCTGAACCCAGCC | chr10:1857 | 83 |
| | | R | | 1215 | |
| S097_long.F | AGCCCTGCACACTCACTTACC | S097_short. | TGGCATTCAGATCATCAGGC | chr18:8563 | 83 |
| | | R | TTCT | 171 | |
| S102_long.F | AACAGTGGCAGCCCTCTTGT | S102_short. | ACACTTGGTTCATGGGGTTG | chr13:2759 | 80 |
| | | R | TG | 7230 | |
| S105_long.F | ACCCCAAGAGGCTTTATAGGGG | S105_short. | CCTTCCCAACGGGTTTGACC | chr6:31106 | 96 |
| | | R | | 893 | |
| S108_long.F | ACACTCCTGCTGCGTGTCTG | S108_short. | TTCCTCCCCACCACTCCCAT | chr12:1132 | 96 |
| | | R | | 02967 | |

### Example 11. Evaluation by Sequencing

### Materials and Methods

The same source material as described in Example 10 was used. The 135 bp DNA fragments were then again serially diluted into the 240 bp DNA fragments at three different concentrations. Additionally, one mixture was prepared with about 1% 240 bp fragments in 99% 135 bp fragments and one mixture contained fragments of equal length (235 bp) at about 1% minor fraction.

Two multiplex amplifications with a) average amplicon length of 47 bp and b) average amplicon length of 122 bp were performed for each sample. Each amplicon targeted one SNP with known population frequency of ˜50%. Primer sets are shown in Table 8. After purification next-generation sequencing adapters and molecular identifiers were added to the amplicons and sequencing was conducted using an Illumina NextSeq500 according to manufacturer's instructions. Resulting sequencing reads were mapped to the human genome (HG19) and allele frequencies for each targeted SNP were recorded with differentiation between short and long amplicons. The mean allele frequencies for each sample were calculated over all informative assays (defined by recipient/donor allele combinations of AA/BB, AA/AB, BB/AA, or BB/AB), where heterozygous (AB) donor genotypes were corrected by a factor 2.

TABLE 8
Primer sequences, amplicon position and length

| Primer | | Primer | | Amplicon | Amplicon |
|---|---|---|---|---|---|
| (forward) | Sequence | (reverse) | Sequence | position (HG19) | Length |
| Short Amplicon Primers | | | | | |
| 1240-Pr1 | GCTGGCACCCAACA | 1240-Pr2 | GCCCTGACACCTCAGCA | chr7:127865816 | 48 |
| | TGCCAA | | TGTG | | |
| 1276-Pr1 | CCGCCGACAGGACA | 1276-Pr2 | CGGACACCCCAAAAGG | chr1:1376214 | 47 |
| | GCTTGT | | CGGA | | |
| 1303-Pr1 | GGCCTGTTTCCCTG | 1303-Pr2 | GGCAAAGCAGCTCTGG | chr16:8339O775 | 48 |
| | GCCCA | | AGTCTG | | |
| 1666-Pr1 | GGGAGCGAAGGAG | 1666-Pr2 | CCAGGGAGAGGCTGTG | chr20:23104218 | 48 |
| | CCCCAC | | TTCCTG | | |
| 1817-Pr1 | CCCTCAAGGGCTCC | 1817-Pr2 | CCAGTCTGGGGACTGA | chr9:130968398 | 48 |
| | TGACCTC | | GCCC | | |
| 2050-Pr1 | GCTGCATTTCCACA | 2050-Pr2 | CCTGTGGGACCCTCGCC | chr10:13360823 | 47 |
| | GGCCCC | | TCT | 7 | |
| 2333-Pr1 | GCGCGGAAGGCCC | 2333-Pr2 | GCCTTTGTAAAAGCTCT | chr15:63779752 | 47 |
| | GAGA | | GCGGGA | | |
| 2350-Pr1 | CAACCCCAGGGCTT | 2350-Pr2 | CGGCGCGTCCTGCGGA | chr1:156499969 | 45 |
| | CAAGTCG | | A | | |
| 633-Pr1 | CCAGACGCACCCTG | 633-Pr2 | TGCCCAGGGAACTGCC | chr10:13288387 | 47 |
| | AGGGAAT | | AGC | 8 | |
| 1054-Pr1 | CCCAGCCTTGCTTCC | 1054-Pr2 | GGCCACCGCCTCTGAT | chr3:12857823 | 48 |
| | AGGGAG | | GCAA | | |
| 1169-Pr1 | CGAAAGCCTGCCAG | 1169-Pr2 | ACCGCCCAAGGCGTCTT | chr17:41994794 | 48 |
| | TTCTGAGC | | CC | | |
| 1187-Pr1 | GCGGCCCCGGCTAG | 1187-Pr2 | CCGGGTCACAAAGGCA | chr9:106856691 | 47 |
| | AGAGT | | GGGAA | | |
| 1373-Pr1 | GTGCAGTGTTCACG | 1373-Pr2 | GCAGGCGCCCGATCCC | chr5:178836180 | 47 |
| | GAAGGCA | | AAT | | |
| 172-Pr1 | GGCTGAGAGCCAG | 172-Pr2 | CGTGAGAGCCCGTTGG | chr20:887521 | 48 |
| | GGGTAGAG | | TCCC | | |
| 1822-Pr1 | CCACCTGGCTCTCCT | 1822-Pr2 | AGGCGGAGACACACAC | chr5:179290845 | 47 |
| | GTGGG | | CTCG | | |
| 1857-Pr1 | ACCGTGCAGGGAG | 1857-Pr2 | CGCTTCCTCCTGGAGTC | chr9:132400480 | 48 |
| | CAGTTGTA | | CGG | | |
| 1879-Pr1 | GGGCGGACCCTGTA | 1879-Pr2 | GGTGGCTGTGCCAGCT | chr16:89842029 | 47 |
| | CCCAAA | | CAAG | | |
| 2472-Pr1 | GCAAGCCCCTGGCC | 2472-Pr2 | GCAGCTGGATGACTTC | chr1:10438687 | 47 |
| | TTTGC | | CCGCT | | |
| 2657-Pr1 | CGAGGGAAGCGGC | 2657-Pr2 | CCGCAGGAGCCCGACA | chr7:2412032 | 43 |
| | ATCCACA | | TTGT | | |
| S2023.F_SH | CCTGACCACCCCTC | S2023.RS | GCCCCGCCAAGCCGTG | chr11:61671956 | 43 |
| OLO_P5 | GGCAC | H0P7 | T | | |
| Long Amplicon Primers | | | | | |
| 1240-Pr1 | GCTGGCACCCAACA | 1240-Pr4 | TTGCACCTCCCAGGCTT | chr7:127865816 | 121 |
| | TGCCAA | | CTTG | | |
| 1276-Pr1 | CCGCCGACAGGACA | 1276-Pr4 | GGCCCAAGCTCTCTGA | chr1:1376214 | 125 |
| | GCTTGT | | ATAAAAGGT | | |
| 1303-Pr1 | GGCCTGTTTCCCTG | 1303-Pr4 | GTGAGCCAACAAGCAT | chr16:83390775 | 123 |
| | GCCCA | | AGAACCTC | | |
| 1666-Pr1 | GGGAGCGAAGGAG | 1666-Pr4 | TGGGACACTGGTCTAA | chr20:23104218 | 123 |
| | CCCCAC | | GGACTACA | | |
| 1817-Pr1 | CCCTCAAGGGCTCC | 1817-Pr4 | TGATGCATTTTCCTGTG | chr9:130968398 | 128 |
| | TGACCTC | | AAGCATTTTC | | |
| 2050-Pr1 | GCTGCATTTCCACA | 2050-Pr4 | GAATTGTCCTCTCTTCT | chr10:13360823 | 125 |
| | GGCCCC | | GCCAGAGA | 7 | |
| 2333-Pr1 | GCGCGGAAGGCCC | 2333-Pr4 | CGCTGGGTTCTTTGTCA | chr15:63779752 | 120 |
| | GAGA | | CAGTGT | | |
| 2350-Pr1 | CAACCCCAGGGCTT | 2350-Pr4 | TGTACAGCCCAGACAC | chr1:156499969 | 122 |
| | CAAGTCG | | CGGAG | | |
| 633-Pr1 | CCAGACGCACCCTG | 633-Pr4 | GCTGGGGGAACTCAGG | chr10:13288387 | 121 |
| | AGGGAAT | | AGCA | 8 | |
| 1054-Pr1 | CCCAGCCTTGCTTCC | 1054-Pr4 | TCCATCCGGATGGTGG | chr3:12857823 | 122 |
| | AGGGAG | | AGGAG | | |
| 1169-Pr1 | CGAAAGCCTGCCAG | 1169-Pr4 | GGGCCGTGGCTGAAGA | chr17:41994794 | 121 |
| | TTCTGAGC | | AGC | | |
| 1187-Pr1 | GCGGCCCCGGCTAG | 1187-Pr4 | CCCAACCGGCCGTGCTC | chr9:106856691 | 117 |
| | AGAGT | | A | | |
| 1373-Pr1 | GTGCAGTGTTCACG | 1373-Pr4 | CCTTGAAACCTCCCTTT | chr5:178836180 | 123 |
| | GAAGGCA | | CCCCG | | |
| 172-Pr1 | GGCTGAGAGCCAG | 172-Pr4 | CAGACCACACTGGATG | chr20:887521 | 125 |
| | GGGTAGAG | | TTTGGAGA | | |
| 1822-Pr1 | CCACCTGGCTCTCCT | 1822-Pr4 | CCACCAGCACCCGAACT | chr5:179290845 | 120 |
| | GTGGG | | GCA | | |
| 1857-Pr1 | ACCGTGCAGGGAG | 1857-Pr4 | CCCAGCGAGTGCCCAG | chr9:132400480 | 119 |
| | CAGTTGTA | | CC | | |
| 1879-Pr1 | GGGCGGACCCTGTA | 1879-Pr4 | TGTCCACTCTGGGACTT | chr16:89842029 | 122 |
| | CCCAAA | | GTGCT | | |
| 2472-Pr1 | GCAAGCCCCTGGCC | 2472-Pr4 | TGCTCAGGCATTCAGG | chr1:10438687 | 124 |
| | TTTGC | | AAAGTATCT | | |
| 2657-Pr1 | CGAGGGAAGCGGC | 2657-Pr4 | CAGAGACAGCTGCTTC | chr7:2412032 | 123 |
| | ATCCACA | | CACTTGT | | |
| S2023.F_SH | CCTGACCACCCCTC | S2023.RL | CACAGTGAAGGTGGCC | chr11:61671956 | 119 |
| OLO_P5 | GGCAC | 0_P7 | GAGG | | |

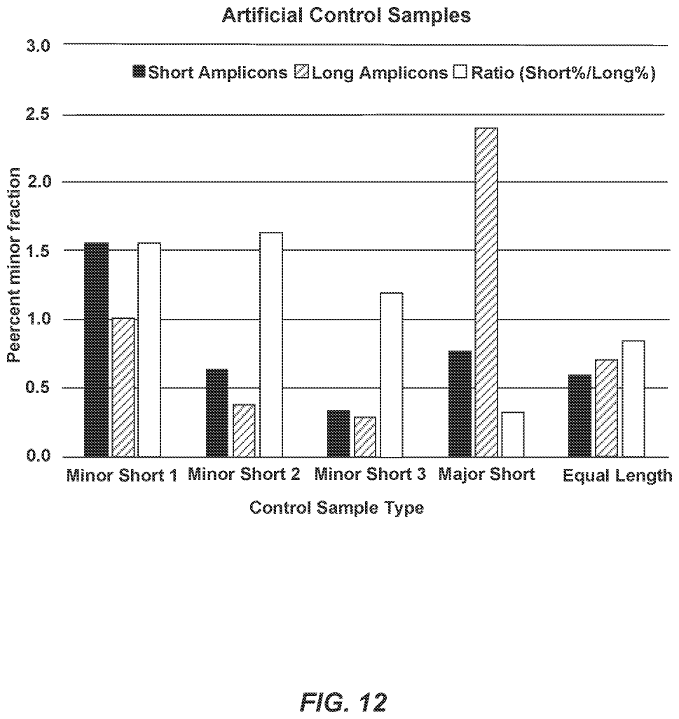

FIG. 12 shows the mean allele frequencies and ratio of short/long amplicons for the different samples. Minor Short1l-3: minor fraction composed of shorter template fragments (135 bp) than major fraction (240 bp); Major short: minor fraction composed of longer template fragments (240 bp) than major fraction (135 bp); Equal length: minor and major fraction had the same length (235 bp)

These data demonstrated that the ratio between short amplicon allelic fractions vs long amplicon allelic fractions were >1 for samples Minor Short1-3 while the ratio was <1 for the sample Major short and close to 1 for the sample Equal LengthB3.

All accession numbers, patents, patent applications, and other published reference materials cited in this specification are hereby incorporated herein by reference in their entirety for their disclosures of the subject matter in whose connection they are cited herein.

1. A method to quantify the amount of a diagnostic cfDNA in a cell-free DNA (cfDNA) sample from a subject using two or more PCR reactions, wherein the diagnostic cfDNA deviates from germline DNA of the subject; the method comprising:
(a) performing a first PCR on cfDNA from the sample that amplifies a first region that comprises a target sequence that differs in germline DNA from the subject and the diagnostic cfDNA to obtain a first amplicon;
(b) performing at least a second PCR on cfDNA from the sample that amplifies a second a second region that comprises a target sequence that differs in germline DNA from the subject and the diagnostic cfDNA to obtain a second amplicon that differs in length from the first amplicon by at least 10 base pairs;
(c) quantifying the yield of the first amplicon that is generated from the diagnostic cfDNA and the yield of the first amplicon that is generated from germline cfDNA;
(d) quantifying the yield of the second amplicon that is generated from the diagnostic cfDNA and the yield of the second amplicon that is generated from germline cfDNA; and
(d) determining the amount of diagnostic cfDNA present in the cfDNA sample.

2. The method of claim 1, wherein the target sequence in (a) and (b) are different sequences.

3. The method of claim 1, wherein the target sequence in (a) and (b) is the same sequence.

4. The method of claim 1, wherein the amount of diagnostic cfDNA determined in (d) is determined as a percentage.

5. The method of claim 1, wherein the amount of diagnostic cfDNA determined in (d) is determined a concentration.

6. The method of claim 1, wherein the subject is a transplant patient and the diagnostic cfDNA is from a donor graft.

7. The method of claim 6, wherein the length of the dd-cfDNA is determined to distinguish between ABMR, TCMR and ATN in kidney recipients.

8. The method of claim 1, wherein the subject is a cancer patient and the diagnostic cfDNA is from tumor cells.

9. The method of claim 1, wherein the subject is a pregnant patient and the diagnostic cfDNA is from fetal cells.

10. The method of claim 1, wherein the patient is a human.

11. The method of claim 1, further comprising an amplification reaction that targets DNA that was added to the blood, serum, or plasma sample prior to extraction.

12. The method of claim 1, wherein the two or more PCR reactions are performed in a multiplex assay.

13. A control sample comprising an artificial target cf DNA that represents cfDNA and an artificial host DNA that represents cellular DNA from the subject, wherein the artificial target in which the artificial target cfDNA is of different length than the artificial host DNA for use the method of claim 1, optionally wherein the control sample is for use as a quality control of PCR methods.

14. (canceled)

15. A control sample comprising of a mixture of at least two non-identical DNA samples, which are sheared to different lengths, wherein the first sample comprises an artificial target cfDNA that represents cfDNA in a subject and the second sample comprises an artificial host DNA that represents cellular DNA from the subject


SEQUENCE LISTING (178 sequences)

SEQ ID NO: 1 - DNA, 19 bp, Artificial Sequence
  Description of Artificial Sequence Synthetic primer
gtatcgtcaaggcactctt

SEQ ID NO: 2 - DNA, 20 bp, Artificial Sequence
  Description of Artificial Sequence Synthetic primer
cctgctgaaaatgactgaat

SEQ ID NO: 3 - DNA, 20 bp, Artificial Sequence
  Description of Artificial Sequence Synthetic primer
cgtccacaaaatgattctga

SEQ ID NO: 4 - DNA, 20 bp, Artificial Sequence
  Description of Artificial Sequence Synthetic primer
ataaggcctgctgaaaatga

SEQ ID NO: 5 - DNA, 18 bp, Artificial Sequence
  Description of Artificial Sequence Synthetic probe
tcttgcctacgccaccag

SEQ ID NO: 6 - DNA, 18 bp, Artificial Sequence
  Description of Artificial Sequence Synthetic probe
tagttggagctgatggcg

SEQ ID NO: 7 - DNA, 21 bp, Artificial Sequence
  Description of Artificial Sequence Synthetic primer
gaggacttggcacaggtgctc

SEQ ID NO: 8 - DNA, 24 bp, Artificial Sequence
  Description of Artificial Sequence Synthetic primer
tctgccctgggagagaaagaacaa

SEQ ID NO: 9 - DNA, 25 bp, Artificial Sequence
  Description of Artificial Sequence Synthetic primer
cacaacttccctaagggactgacat

SEQ ID NO: 10 - DNA, 25 bp, Artificial Sequence
  Description of Artificial Sequence Synthetic primer
ggactcttgccagtttccatgacaa

SEQ ID NO: 11 - DNA, 23 bp, Artificial Sequence
  Description of Artificial Sequence Synthetic primer
gtttctgaggctcggttttcgct

SEQ ID NO: 12 - DNA, 21 bp, Artificial Sequence
  Description of Artificial Sequence Synthetic primer
aaggcaggacttctccaccca

SEQ ID NO: 13 - DNA, 27 bp, Artificial Sequence
  Description of Artificial Sequence Synthetic primer
acatccatggaatacttctcagcaaca

SEQ ID NO: 14 - DNA, 24 bp, Artificial Sequence
  Description of Artificial Sequence Synthetic primer
ttgcacactcacgtttgggatact

SEQ ID NO: 15 - DNA, 26 bp, Artificial Sequence
  Description of Artificial Sequence Synthetic primer
ccaaagtggtaggattacaggcatga

SEQ ID NO: 16 - DNA, 26 bp, Artificial Sequence
  Description of Artificial Sequence Synthetic primer
gtctgaattttactcctggctctgca

SEQ ID NO: 17 - DNA, 28 bp, Artificial Sequence
  Description of Artificial Sequence Synthetic primer
tcagttccttcaccactgttatttgttc

SEQ ID NO: 18 - DNA, 22 bp, Artificial Sequence
  Description of Artificial Sequence Synthetic primer
gcgaagttggagagggttgggg

SEQ ID NO: 19 - DNA, 21 bp, Artificial Sequence
  Description of Artificial Sequence Synthetic primer
cacgcccttctgagtcccaca

SEQ ID NO: 20 - DNA, 23 bp, Artificial Sequence
  Description of Artificial Sequence Synthetic primer
atgctggcataccctcctgtact

SEQ ID NO: 21 - DNA, 27 bp, Artificial Sequence
  Description of Artificial Sequence Synthetic primer
gctattgtatgctctatgctcagcaca

SEQ ID NO: 22 - DNA, 19 bp, Artificial Sequence
  Description of Artificial Sequence Synthetic primer
aggcgccggcagcaggtgc

SEQ ID NO: 23 - DNA, 22 bp, Artificial Sequence
  Description of Artificial Sequence Synthetic primer
ggaaggaagtgcaggagggctg

SEQ ID NO: 24 - DNA, 26 bp, Artificial Sequence
  Description of Artificial Sequence Synthetic primer
catgatgctcacaaagacattcctcc

SEQ ID NO: 25 - DNA, 25 bp, Artificial Sequence
  Description of Artificial Sequence Synthetic primer
gaaacatctagacgcgcaatgacac

SEQ ID NO: 26 - DNA, 23 bp, Artificial Sequence
  Description of Artificial Sequence Synthetic primer
cagccgggaggaaagaaaccttt

SEQ ID NO: 27 - DNA, 24 bp, Artificial Sequence
  Description of Artificial Sequence Synthetic primer
aggctataaacactgggatggggg

SEQ ID NO: 28 - DNA, 24 bp, Artificial Sequence
  Description of Artificial Sequence Synthetic primer
ggaaaaggaattcctggcagaggg

SEQ ID NO: 29 - DNA, 22 bp, Artificial Sequence
  Description of Artificial Sequence Synthetic primer
aggtcctcaagctgtgcaactg

SEQ ID NO: 30 - DNA, 26 bp, Artificial Sequence
  Description of Artificial Sequence Synthetic primer
gcttaggtttgaggtagcacagagga

SEQ ID NO: 31 - DNA, 21 bp, Artificial Sequence
  Description of Artificial Sequence Synthetic primer
ccctcctccatcaggtgctgg

SEQ ID NO: 32 - DNA, 24 bp, Artificial Sequence
  Description of Artificial Sequence Synthetic primer
gttgtggccttatctttggcccta

SEQ ID NO: 33 - DNA, 25 bp, Artificial Sequence
  Description of Artificial Sequence Synthetic primer
ctttataggggaggagtggaggagg

SEQ ID NO: 34 - DNA, 23 bp, Artificial Sequence
  Description of Artificial Sequence Synthetic primer
actccccacatatatgctcccca

SEQ ID NO: 35 - DNA, 23 bp, Artificial Sequence
  Description of Artificial Sequence Synthetic primer
gctctctaagtggagacgggtcc

SEQ ID NO: 36 - DNA, 25 bp, Artificial Sequence
  Description of Artificial Sequence Synthetic primer
ggtctcctttcatttccccaaatgc

SEQ ID NO: 37 - DNA, 24 bp, Artificial Sequence
  Description of Artificial Sequence Synthetic primer
tctcatctgtaaaagggggtggtg

SEQ ID NO: 38 - DNA, 19 bp, Artificial Sequence
  Description of Artificial Sequence Synthetic primer
aacacgccgggagccctgc

SEQ ID NO: 39 - DNA, 23 bp, Artificial Sequence
  Description of Artificial Sequence Synthetic primer
ggtcgccgaatagtccatttcac

SEQ ID NO: 40 - DNA, 23 bp, Artificial Sequence
  Description of Artificial Sequence Synthetic primer
cccaaggtggcttttgagacaag

SEQ ID NO: 41 - DNA, 27 bp, Artificial Sequence
  Description of Artificial Sequence Synthetic primer
cagcatagttctctggaaattcatcca

SEQ ID NO: 42 - DNA, 25 bp, Artificial Sequence
  Description of Artificial Sequence Synthetic primer
agaaccccagtaaggaatggagaaa

SEQ ID NO: 43 - DNA, 20 bp, Artificial Sequence
  Description of Artificial Sequence Synthetic primer
caaacttggcaaggcacggt

SEQ ID NO: 44 - DNA, 28 bp, Artificial Sequence
  Description of Artificial Sequence Synthetic primer
gggaagggacttgattacatagcttatc

SEQ ID NO: 45 - DNA, 26 bp, Artificial Sequence
  Description of Artificial Sequence Synthetic primer
ggagctaggatattgctagagtggag

SEQ ID NO: 46 - DNA, 23 bp, Artificial Sequence
  Description of Artificial Sequence Synthetic primer
agtaccaagaccccgacccttaa

SEQ ID NO: 47 - DNA, 23 bp, Artificial Sequence
  Description of Artificial Sequence Synthetic primer
tgttggaaaggacagcaggagac

SEQ ID NO: 48 - DNA, 22 bp, Artificial Sequence
  Description of Artificial Sequence Synthetic primer
gcgtgtggacagtgaaggtgtg

SEQ ID NO: 49 - DNA, 22 bp, Artificial Sequence
  Description of Artificial Sequence Synthetic primer
tgattcacctgcactgcttccc

SEQ ID NO: 50 - DNA, 26 bp, Artificial Sequence
  Description of Artificial Sequence Synthetic primer
gctctctaagcattaggcattactgc

SEQ ID NO: 51 - DNA, 22 bp, Artificial Sequence
  Description of Artificial Sequence Synthetic primer
ccagtcaaggcctctgctctca

SEQ ID NO: 52 - DNA, 21 bp, Artificial Sequence
  Description of Artificial Sequence Synthetic primer
ggagtggaggccagaagtccc

SEQ ID NO: 53 - DNA, 19 bp, Artificial Sequence
  Description of Artificial Sequence Synthetic primer
ggcggaatcgggaggccac

SEQ ID NO: 54 - DNA, 26 bp, Artificial Sequence
  Description of Artificial Sequence Synthetic primer
aggtgcttgtgaggattaactgacat

SEQ ID NO: 55 - DNA, 24 bp, Artificial Sequence
  Description of Artificial Sequence Synthetic primer
agctgagtttcaaggcttgtacac

SEQ ID NO: 56 - DNA, 21 bp, Artificial Sequence
  Description of Artificial Sequence Synthetic primer
ctccctgcctatgctcaggca

SEQ ID NO: 57 - DNA, 23 bp, Artificial Sequence
  Description of Artificial Sequence Synthetic primer
agaccttcaaaccaccttgtggc

SEQ ID NO: 58 - DNA, 19 bp, Artificial Sequence
  Description of Artificial Sequence Synthetic primer
gaacccagccacagctgca

SEQ ID NO: 59 - DNA, 22 bp, Artificial Sequence
  Description of Artificial Sequence Synthetic primer
gcttctgatcctgcagggaaga

SEQ ID NO: 60 - DNA, 25 bp, Artificial Sequence
  Description of Artificial Sequence Synthetic primer
cctcttgtttgaggcacatcctaca

SEQ ID NO: 61 - DNA, 20 bp, Artificial Sequence
  Description of Artificial Sequence Synthetic primer
attcctgtgccactgggctg

SEQ ID NO: 62 - DNA, 20 bp, Artificial Sequence
  Description of Artificial Sequence Synthetic primer
gtctgcatggtcccagctgg

SEQ ID NO: 63 - DNA, 25 bp, Artificial Sequence
  Description of Artificial Sequence Synthetic primer
tcaatcctcacaacttccctaaggg

SEQ ID NO: 64 - DNA, 24 bp, Artificial Sequence
  Description of Artificial Sequence Synthetic primer
tccagcagaggaaatagtacttgc

SEQ ID NO: 65 - DNA, 21 bp, Artificial Sequence
  Description of Artificial Sequence Synthetic primer
gtctctgggggtctgttggcc

SEQ ID NO: 66 - DNA, 24 bp, Artificial Sequence
  Description of Artificial Sequence Synthetic primer
gatcaactcctgaagagactccgt

SEQ ID NO: 67 - DNA, 24 bp, Artificial Sequence
  Description of Artificial Sequence Synthetic primer
tcttgtcgaggctgccctgaaagg

SEQ ID NO: 68 - DNA, 20 bp, Artificial Sequence
  Description of Artificial Sequence Synthetic primer
caaagagctcraaccccaag

SEQ ID NO: 69 - DNA, 26 bp, Artificial Sequence
  Description of Artificial Sequence Synthetic primer
tggttaaactgtagtacatccatgga

SEQ ID NO: 70 - DNA, 20 bp, Artificial Sequence
  Description of Artificial Sequence Synthetic primer
cagcctctggttccaggcct

SEQ ID NO: 71 - DNA, 20 bp, Artificial Sequence
  Description of Artificial Sequence Synthetic primer
gccaccttagcctcccaaag

SEQ ID NO: 72 - DNA, 25 bp, Artificial Sequence
  Description of Artificial Sequence Synthetic primer
agaaagaaagaagcagggaagggac

SEQ ID NO: 73 - DNA, 20 bp, Artificial Sequence
  Description of Artificial Sequence Synthetic primer
accctgaccctcagttcctt

SEQ ID NO: 74 - DNA, 20 bp, Artificial Sequence
  Description of Artificial Sequence Synthetic primer
atgaagagtaagcggggccg

SEQ ID NO: 75 - DNA, 19 bp, Artificial Sequence
  Description of Artificial Sequence Synthetic primer
gcctctgccatatcctcac

SEQ ID NO: 76 - DNA, 22 bp, Artificial Sequence
  Description of Artificial Sequence Synthetic primer
tggcccagttagaaggtgtgga

SEQ ID NO: 77 - DNA, 21 bp, Artificial Sequence
  Description of Artificial Sequence Synthetic primer
gggcctcagttctagacgagt

SEQ ID NO: 78 - DNA, 21 bp, Artificial Sequence
  Description of Artificial Sequence Synthetic primer
cagggagtgctttactgaggc

SEQ ID NO: 79 - DNA, 20 bp, Artificial Sequence
  Description of Artificial Sequence Synthetic primer
tttgcacttgacgcaccagc

SEQ ID NO: 80 - DNA, 27 bp, Artificial Sequence
  Description of Artificial Sequence Synthetic primer
ccccaaactaagtacctaatcactcgt

SEQ ID NO: 81 - DNA, 21 bp, Artificial Sequence
  Description of Artificial Sequence Synthetic primer
acacacacacacgcaattcgg

SEQ ID NO: 82 - DNA, 20 bp, Artificial Sequence
  Description of Artificial Sequence Synthetic primer
gtctccctccccaaaggtgc

SEQ ID NO: 83 - DNA, 24 bp, Artificial Sequence
  Description of Artificial Sequence Synthetic primer
ggcatctgaattcaagctttggtc

SEQ ID NO: 84 - DNA, 20 bp, Artificial Sequence
  Description of Artificial Sequence Synthetic primer
gttgaacgtccacagaagga

SEQ ID NO: 85 - DNA, 24 bp, Artificial Sequence
  Description of Artificial Sequence Synthetic primer
tttatttaaatgactgtccaggtc

SEQ ID NO: 86 - DNA, 20 bp, Artificial Sequence
  Description of Artificial Sequence Synthetic primer
ctggggcagagtggagagtc

SEQ ID NO: 87 - DNA, 21 bp, Artificial Sequence
  Description of Artificial Sequence Synthetic primer
agccctgcacactcacttacc

SEQ ID NO: 88 - DNA, 20 bp, Artificial Sequence
  Description of Artificial Sequence Synthetic primer
aacagtggcagccctcttgt

SEQ ID NO: 89 - DNA, 22 bp, Artificial Sequence
  Description of Artificial Sequence Synthetic primer
accccaagaggctttatagggg

SEQ ID NO: 90 - DNA, 20 bp, Artificial Sequence
  Description of Artificial Sequence Synthetic primer
acactcctgctgcgtgtctg

SEQ ID NO: 91 - DNA, 21 bp, Artificial Sequence
  Description of Artificial Sequence Synthetic primer
agtgggagggaggtacagtga

SEQ ID NO: 92 - DNA, 20 bp, Artificial Sequence
  Description of Artificial Sequence Synthetic primer
agccacctggtctcctttca

SEQ ID NO: 93 - DNA, 21 bp, Artificial Sequence
  Description of Artificial Sequence Synthetic primer
agaggaaggactcccaggggg

SEQ ID NO: 94 - DNA, 20 bp, Artificial Sequence
  Description of Artificial Sequence Synthetic primer
agggagggatggagagggac

SEQ ID NO: 95 - DNA, 18 bp, Artificial Sequence
  Description of Artificial Sequence Synthetic primer
acagagccggccggtcgc

SEQ ID NO: 96 - DNA, 18 bp, Artificial Sequence
  Description of Artificial Sequence Synthetic primer
tgtgggcaaggcaggact

SEQ ID NO: 97 - DNA, 21 bp, Artificial Sequence
  Description of Artificial Sequence Synthetic primer
accttttgggactggctttct

SEQ ID NO: 98 - DNA, 23 bp, Artificial Sequence
  Description of Artificial Sequence Synthetic primer
ggagaatcccagaagcaggctga

SEQ ID NO: 99 - DNA, 30 bp, Artificial Sequence
  Description of Artificial Sequence Synthetic primer
agggtgactgtattaattattgttcaaact

SEQ ID NO: 100 - DNA, 22 bp, Artificial Sequence
  Description of Artificial Sequence Synthetic primer
tggagctaaaatgagcctgcgt

SEQ ID NO: 101 - DNA, 25 bp, Artificial Sequence
  Description of Artificial Sequence Synthetic primer
aagagcccttataaggtgtgagaaa

SEQ ID NO: 102 - DNA, 20 bp, Artificial Sequence
  Description of Artificial Sequence Synthetic primer
cggacccatttcacccacca

SEQ ID NO: 103 - DNA, 19 bp, Artificial Sequence
  Description of Artificial Sequence Synthetic primer
aggtcggatgttggaaagg

SEQ ID NO: 104 - DNA, 20 bp, Artificial Sequence
  Description of Artificial Sequence Synthetic primer
cggccacccatcctggagat

SEQ ID NO: 105 - DNA, 20 bp, Artificial Sequence
  Description of Artificial Sequence Synthetic primer
gtttccgtgaagtaggcgct

SEQ ID NO: 106 - DNA, 20 bp, Artificial Sequence
  Description of Artificial Sequence Synthetic primer
actcaaacacggagctgggc

SEQ ID NO: 107 - DNA, 21 bp, Artificial Sequence
  Description of Artificial Sequence Synthetic primer
ccgaggcagaggaaggaagtg

SEQ ID NO: 108 - DNA, 20 bp, Artificial Sequence
  Description of Artificial Sequence Synthetic primer
ccaaggggagcatccaccat

SEQ ID NO: 109 - DNA, 20 bp, Artificial Sequence
  Description of Artificial Sequence Synthetic primer
atgagctgaggtgggtgctg

SEQ ID NO: 110 - DNA, 20 bp, Artificial Sequence
  Description of Artificial Sequence Synthetic primer
gccaacctcaaggggcagtt

SEQ ID NO: 111 - DNA, 24 bp, Artificial Sequence
  Description of Artificial Sequence Synthetic primer
ttcttctagttggtctggtaggct

SEQ ID NO: 112 - DNA, 18 bp, Artificial Sequence
  Description of Artificial Sequence Synthetic primer
ggctgctcagcctccctg

SEQ ID NO: 113 - DNA, 21 bp, Artificial Sequence
  Description of Artificial Sequence Synthetic primer
tttcacagaccttcaaaccac

SEQ ID NO: 114 - DNA, 20 bp, Artificial Sequence
  Description of Artificial Sequence Synthetic primer
atccacctctgaacccagcc

SEQ ID NO: 115 - DNA, 24 bp, Artificial Sequence
  Description of Artificial Sequence Synthetic primer
tggcattcagatcatcaggcttct

SEQ ID NO: 116 - DNA, 22 bp, Artificial Sequence
  Description of Artificial Sequence Synthetic primer
acacttggttcatggggttgtg

SEQ ID NO: 117 - DNA, 20 bp, Artificial Sequence
  Description of Artificial Sequence Synthetic primer
ccttcccaacgggtttgacc

SEQ ID NO: 118 - DNA, 20 bp, Artificial Sequence
  Description of Artificial Sequence Synthetic primer
ttcctccccaccactcccat

SEQ ID NO: 119 - DNA, 20 bp, Artificial Sequence
  Description of Artificial Sequence Synthetic primer
gctggcacccaacatgccaa

SEQ ID NO: 120 - DNA, 20 bp, Artificial Sequence
  Description of Artificial Sequence Synthetic primer
ccgccgacaggacagcttgt

SEQ ID NO: 121 - DNA, 19 bp, Artificial Sequence
  Description of Artificial Sequence Synthetic primer
ggcctgtttccctggccca

SEQ ID NO: 122 - DNA, 19 bp, Artificial Sequence
  Description of Artificial Sequence Synthetic primer
gggagcgaaggagccccac

SEQ ID NO: 123 - DNA, 21 bp, Artificial Sequence
  Description of Artificial Sequence Synthetic primer
ccctcaagggctcctgacctc

SEQ ID NO: 124 - DNA, 20 bp, Artificial Sequence
  Description of Artificial Sequence Synthetic primer
gctgcatttccacaggcccc

SEQ ID NO: 125 - DNA, 17 bp, Artificial Sequence
  Description of Artificial Sequence Synthetic primer
gcgcggaaggcccgaga

SEQ ID NO: 126 - DNA, 21 bp, Artificial Sequence
  Description of Artificial Sequence Synthetic primer
caaccccagggcttcaagtcg

SEQ ID NO: 127 - DNA, 21 bp, Artificial Sequence
  Description of Artificial Sequence Synthetic primer
ccagacgcaccctgagggaat

SEQ ID NO: 128 - DNA, 21 bp, Artificial Sequence
  Description of Artificial Sequence Synthetic primer
cccagccttgcttccagggag

SEQ ID NO: 129 - DNA, 22 bp, Artificial Sequence
  Description of Artificial Sequence Synthetic primer
cgaaagcctgccagttctgagc

SEQ ID NO: 130 - DNA, 19 bp, Artificial Sequence
  Description of Artificial Sequence Synthetic primer
gcggccccggctagagagt

SEQ ID NO: 131 - DNA, 21 bp, Artificial Sequence
  Description of Artificial Sequence Synthetic primer
gtgcagtgttcacggaaggca

SEQ ID NO: 132 - DNA, 21 bp, Artificial Sequence
  Description of Artificial Sequence Synthetic primer
ggctgagagccaggggtagag

SEQ ID NO: 133 - DNA, 20 bp, Artificial Sequence
  Description of Artificial Sequence Synthetic primer
ccacctggctctcctgtggg

SEQ ID NO: 134 - DNA, 21 bp, Artificial Sequence
  Description of Artificial Sequence Synthetic primer
accgtgcagggagcagttgta

SEQ ID NO: 135 - DNA, 20 bp, Artificial Sequence
  Description of Artificial Sequence Synthetic primer
gggcggaccctgtacccaaa

SEQ ID NO: 136 - DNA, 19 bp, Artificial Sequence
  Description of Artificial Sequence Synthetic primer
gcaagcccctggcctttgc

SEQ ID NO: 137 - DNA, 20 bp, Artificial Sequence
  Description of Artificial Sequence Synthetic primer
cgagggaagcggcatccaca

SEQ ID NO: 138 - DNA, 19 bp, Artificial Sequence
  Description of Artificial Sequence Synthetic primer
cctgaccacccctcggcac

SEQ ID NO: 139 - DNA, 21 bp, Artificial Sequence
  Description of Artificial Sequence Synthetic primer
gccctgacacctcagcatgtg

SEQ ID NO: 140 - DNA, 20 bp, Artificial Sequence
  Description of Artificial Sequence Synthetic primer
cggacaccccaaaaggcgga

SEQ ID NO: 141 - DNA, 22 bp, Artificial Sequence
  Description of Artificial Sequence Synthetic primer
ggcaaagcagctctggagtctg

SEQ ID NO: 142 - DNA, 22 bp, Artificial Sequence
  Description of Artificial Sequence Synthetic primer
ccagggagaggctgtgttcctg

SEQ ID NO: 143 - DNA, 20 bp, Artificial Sequence
  Description of Artificial Sequence Synthetic primer
ccagtctggggactgagccc

SEQ ID NO: 144 - DNA, 20 bp, Artificial Sequence
  Description of Artificial Sequence Synthetic primer
cctgtgggaccctcgcctct

SEQ ID NO: 145 - DNA, 23 bp, Artificial Sequence
  Description of Artificial Sequence Synthetic primer
gcctttgtaaaagctctgcggga

SEQ ID NO: 146 - DNA, 17 bp, Artificial Sequence
  Description of Artificial Sequence Synthetic primer
cggcgcgtcctgcggaa

SEQ ID NO: 147 - DNA, 19 bp, Artificial Sequence
  Description of Artificial Sequence Synthetic primer
tgcccagggaactgccagc

SEQ ID NO: 148 - DNA, 20 bp, Artificial Sequence
  Description of Artificial Sequence Synthetic primer
ggccaccgcctctgatgcaa

SEQ ID NO: 149 - DNA, 19 bp, Artificial Sequence
  Description of Artificial Sequence Synthetic primer
accgcccaaggcgtcttcc

SEQ ID NO: 150 - DNA, 21 bp, Artificial Sequence
  Description of Artificial Sequence Synthetic primer
ccgggtcacaaaggcagggaa

SEQ ID NO: 151 - DNA, 19 bp, Artificial Sequence
  Description of Artificial Sequence Synthetic primer
gcaggcgcccgatcccaat

SEQ ID NO: 152 - DNA, 20 bp, Artificial Sequence
  Description of Artificial Sequence Synthetic primer
cgtgagagcccgttggtccc

SEQ ID NO: 153 - DNA, 20 bp, Artificial Sequence
  Description of Artificial Sequence Synthetic primer
aggcggagacacacacctcg

SEQ ID NO: 154 - DNA, 20 bp, Artificial Sequence
  Description of Artificial Sequence Synthetic primer
cgcttcctcctggagtccgg

SEQ ID NO: 155 - DNA, 20 bp, Artificial Sequence
  Description of Artificial Sequence Synthetic primer
ggtggctgtgccagctcaag

SEQ ID NO: 156 - DNA, 21 bp, Artificial Sequence
  Description of Artificial Sequence Synthetic primer
gcagctggatgacttcccgct

SEQ ID NO: 157 - DNA, 20 bp, Artificial Sequence
  Description of Artificial Sequence Synthetic primer
ccgcaggagcccgacattgt

SEQ ID NO: 158 - DNA, 17 bp, Artificial Sequence
  Description of Artificial Sequence Synthetic primer
gccccgccaagccgtgt

SEQ ID NO: 159 - DNA, 21 bp, Artificial Sequence
  Description of Artificial Sequence Synthetic primer
ttgcacctcccaggcttcttg

SEQ ID NO: 160 - DNA, 25 bp, Artificial Sequence
  Description of Artificial Sequence Synthetic primer
ggcccaagctctctgaataaaaggt

SEQ ID NO: 161 - DNA, 24 bp, Artificial Sequence
  Description of Artificial Sequence Synthetic primer
gtgagccaacaagcatagaacctc

SEQ ID NO: 162 - DNA, 24 bp, Artificial Sequence
  Description of Artificial Sequence Synthetic primer
tgggacactggtctaaggactaca

SEQ ID NO: 163 - DNA, 27 bp, Artificial Sequence
  Description of Artificial Sequence Synthetic primer
tgatgcattttcctgtgaagcattttc

SEQ ID NO: 164 - DNA, 25 bp, Artificial Sequence
  Description of Artificial Sequence Synthetic primer
gaattgtcctctcttctgccagaga

SEQ ID NO: 165 - DNA, 23 bp, Artificial Sequence
  Description of Artificial Sequence Synthetic primer
cgctgggttctttgtcacagtgt

SEQ ID NO: 166 - DNA, 21 bp, Artificial Sequence
  Description of Artificial Sequence Synthetic primer
tgtacagcccagacaccggag

SEQ ID NO: 167 - DNA, 20 bp, Artificial Sequence
  Description of Artificial Sequence Synthetic primer
gctgggggaactcaggagca

SEQ ID NO: 168 - DNA, 21 bp, Artificial Sequence
  Description of Artificial Sequence Synthetic primer
tccatccggatggtggaggag

SEQ ID NO: 169 - DNA, 19 bp, Artificial Sequence
  Description of Artificial Sequence Synthetic primer
gggccgtggctgaagaagc

SEQ ID NO: 170 - DNA, 18 bp, Artificial Sequence
  Description of Artificial Sequence Synthetic primer
cccaaccggccgtgctca

SEQ ID NO: 171 - DNA, 22 bp, Artificial Sequence
  Description of Artificial Sequence Synthetic primer
ccttgaaacctccctttccccg

SEQ ID NO: 172 - DNA, 24 bp, Artificial Sequence
  Description of Artificial Sequence Synthetic primer
cagaccacactggatgtttggaga

SEQ ID NO: 173 - DNA, 20 bp, Artificial Sequence
  Description of Artificial Sequence Synthetic primer
ccaccagcacccgaactgca

SEQ ID NO: 174 - DNA, 18 bp, Artificial Sequence
  Description of Artificial Sequence Synthetic primer
cccagcgagtgcccagcc

SEQ ID NO: 175 - DNA, 22 bp, Artificial Sequence
  Description of Artificial Sequence Synthetic primer
tgtccactctgggacttgtgct

SEQ ID NO: 176 - DNA, 25 bp, Artificial Sequence
  Description of Artificial Sequence Synthetic primer
tgctcaggcattcaggaaagtatct

SEQ ID NO: 177 - DNA, 23 bp, Artificial Sequence
  Description of Artificial Sequence Synthetic primer
cagagacagctgcttccacttgt

SEQ ID NO: 178 - DNA, 20 bp, Artificial Sequence
  Description of Artificial Sequence Synthetic primer
cacagtgaaggtggccgagg